(19) United States
(12) Patent Application Publication
(10) Pub. No.: US 2006/0178700 A1
(43) Pub. Date: Aug. 10, 2006

(54) Medical device suitable for use in treatment of a valve

(76) Inventor: Martin Quinn, Blackrock (IE)

Correspondence Address: JACOBSON HOLMAN PLLC, 400 SEVENTH STREET N.W., SUITE 600, WASHINGTON, DC, 20004, US

(21) Appl. No.: 11/300,580

(22) Filed: Dec. 15, 2005

(30) Foreign Application Priority Data
Dec. 15, 2004 (IE) S2004/0841

(51) Int. Cl. A61B 17/08

(52) U.S. Cl. 606/213

(57) ABSTRACT

A medical device (1210) comprises a generally cylindrical treatment element (1220) for location between a pair of valve leaflets (1212) situated between an atrium (1214) and a ventricle (1216) of a heart. The treatment element (1220) supports the valve leaflets (1212) at the region of co-aptation of the valve leaflets (1212) and occludes the valve opening to resist fluid flow in the retrograde direction through the valve opening. The device (1210) comprises a support (1222) to support the treatment element (1210) at the region of co-aptation of the valve leaflets (1212). The support has an anchor (1224) and a tether (1226), the tether (1226) being provided at the end of a guide wire (1228) which is initially utilised in the percutaneous insertion of the treatment element (1220). The anchor (1224) is secured, in use, to a septal wall (1230), while the guide wire (1228) exits the atrium (1214) through a vein adjacent a rear wall (1224) thereof. The treatment element (1220) includes a remotely actuatable clamp therein, in order to allow the treatment element (1220) to be secured to the guide wire (1228) or the tether (1226).

### INTRODUCTION

This invention relates to a surgical device and method. In particular this invention relates to a surgical device for treating leaking heart valves, such as the atrioventricular valves, and to a surgical method for treating such heart valves in order to reduce or eliminate leakage therefrom.

The heart contains four valves, two semilunar, the aortic and pulmonary valves, and two atrioventricular (AV) valves, the mitral and tricuspid valves. The heart fills with blood from the lungs and body when the AV valves are open. When the heart pumps or contracts, the AV valves close and prevent the blood from regurgitating backwards. The semilunar valves open when the heart pumps allowing the blood to flow into the aorta and main pulmonary artery.

Dysfunction of the cardiac AV valves is common and can have profound clinical consequences. Failure of the AV valves to prevent regurgitation leads to an increase in the pressure of blood in the lungs or liver and reduces forward blood flow. Valvular dysfunction either results from a defect in the valve leaflet or supporting structure, or dilation of the fibrous ring supporting the valve. These factors lead to a failure of valve leaflets to meet one another, known as co-aptation, allowing the blood to travel in the wrong direction.

Conventional treatment of leaking AV valves often involves replacement or operative repair of the valves. These treatments are considerable surgical operations requiring cardiopulmonary bypass and are associated with significant morbidity. In many instances patients are too sick or too frail to undergo these operations and hospital stays and recovery phases after such operations are prolonged.

Percutaneous techniques of valve repair have the advantage of being significantly less traumatic for the patient. During such procedures the valve repair is performed from within the heart, accessing the heart through a vein in the neck or the groin. Percutaneous procedures are performed under local anaesthetic and the incisions required to perform the procedures are extremely small. In addition, procedural times and recovery phases are also expected to be significantly less. Current attempts at percutaneous repair of leaking heart valves are based on two techniques, the first being the insertion of a mitral valve support structure into a large cardiac vein known as the coronary sinus, and the second being the insertion of a stitch or clip into the mitral valve leaflets to hold them together.

This invention is aimed at providing an alternative surgical device and method for use in treatment of a valve. In particular this invention is aimed at providing a surgical device and method for use in the percutaneous treatment or repair of leaking heart valves.

### STATEMENTS OF INVENTION

According to the invention there is provided a medical device suitable for use in treatment of a valve, the device comprising a treatment element configured to be located at the region at co-aptation of leaflets of a valve to resist fluid flow in a first direction through an opening of the valve.

The treatment element may act as a support element to at least partially support at least one valve leaflet at the region of co-aptation of the valve leaflets. The treatment element may act as an occluder element to at least partially occlude a valve opening.

By supporting the valve leaflets at the region of co-aptation and/or occluding the valve opening, the medical device of the invention is suitable for use in treatment of a number of defects in an atrioventicular valve, such as valve prolapse, or annular dilation of a valve, or restriction of a valve.

In one case the device is configured for use in treatment of a unidirectional valve. The treatment element may be configured to facilitate fluid flow in a second direction through a valve opening. The first direction may be a retrograde direction. The second direction may be a forward direction.

In one embodiment the treatment element when deployed, is shaped and dimensioned to permit unidirectional flow of fluid therepast.

The treatment element may be configured to be urged towards a valve opening by fluid flow. The treatment element may be configured to be urged towards a valve opening by fluid flow in the first direction. The treatment element may be configured to be urged towards a valve opening by fluid flow in the second direction. The treatment element may be shaped to be urged towards a valve opening by fluid flow.

By arranging the fluid flow to urge the treatment element towards the valve opening, this arrangement assists in preventing the treatment element from moving into the ventricle by an excessive amount or fully into the ventricle. The treatment element may be configured wherein fluid flow urges the treatment element in a direction from the ventricle towards the atrium.

The treatment element may be at least partially substantially cylindrically shaped. The treatment element may be at least partially substantially frusto-conically shaped. The apex end of the frusto-cone may be configured to point substantially towards a valve opening. The treatment element may be at least partially substantially diamond shaped. The treatment element may be at least partially substantially crescent shaped. The concave portion of the crescent may be configured to face substantially towards a valve opening. The crescent shape for the treatment element may be particularly suitable for use with a mitral valve which has a normally crescent shaped opening. The treatment element may comprise a ring element. The treatment element may comprise a disc element.

The treatment element may in another case have an arrangement of three arms protruding radially outwardly from a central body. Such a three-arm shape may be particularly suitable for use with a tricuspid valve which normally has an opening which is similarly shaped.

The treatment element may be formed in a range of dimensions to suit the particular anatomy of a patient.

In one emodiement the treatment element comprises at least one arm. The arm may be configured to protrude substantially laterally relative to a valve opening. The arm may taper inwardly in the lateral direction away from a valve opening.

The side arm fins of the treatment element may assist in ensuring that retrograde blood flow, which occurs upon contraction of the heart, correctly results in effective closure of the region/space which would normally be closed by a healthy valve. The side arm fins may assist in ensuring that the treatment element is correctly positioned at the region of co-aptation of the valve leaflets.

In one case the treatment element is engageable with at least one leaflet of a valve.

The treatment element may be movable between a collapsed configuration and an expanded configuration. In the expanded configuration the treatment element may be engageable with a valve leaflet. In the expanded configuration the treatment element may be sealingly engageable with a valve leaflet. In the collapsed configuration the treatment element may be deliverable through a vasculature to a treatment site.

In one case the treatment element is engageable with a valve leaflet which is movable between a closed configuration and an open configuration. In the closed configuration the treatment element may be engageable with a valve leaflet. In the closed configuration the treatment element may be sealingly engageable with a valve leaflet. The treatment element may comprise a plug element. In the closed configuration the treatment element may be configured to prevent fluid flow through a valve opening. In the open configuration the treatment element may be spaced-apart from the region of co-aptation of the valve leaflets. In the open configuration the treatment element may be configured to resist fluid flow in the first direction through a valve opening. In the open configuration the treatment element may be configured to facilitate fluid flow in the second direction through a valve opening.

In one case the treatment element is engageable with a valve leaflet at an engagement region spaced substantially from an annulus of the valve. The treatment element may be engageable with a valve leaflet at the region of co-aptation of the valve leaflets. The treatment element may be engageable with a valve leaflet at an engagement region in proximity to or within the valve opening.

In one embodiment the treatment element comprises a contact part for engaging with a valve leaflet. The treatment element may comprise a base part. The treatment element may comprise at least one support part for supporting the contact part relative to the base part. The contact part may comprise a membrane, or a mesh, or a weave, or a porous or a micro-porous surface.

In one case the treatment element is configured to be located adjacent an interface between at least a pair of valve leaflets. The treatment element may be configured to at least partially prevent leakage from the interface.

In one embodiment the treatment element has a substantially fluid impermeable contact surface for location adjacent the interface between at least a pair of valve leaflets such that the treatment element at least partially prevents leakage from said interface. The contact surface may be substantially circular, or conical, or cylindrical.

In one case the device comprises at least one support element to support the treatment element at the region of co-aptation of the valve leaflets. The support element may be configured to support the treatment element in a location adjacent to a valve opening. The support element may be configured to support the treatment element in a location externally of a valve opening. The support element may be configured to support the treatment element extending at least partially through a valve opening.

It will be appreciated that movement of the heart, for example during the cardiac beating cycle, may result in the treatment element moving relative to the valve leaflets. By extending the treatment element at least partially through the valve opening, this arrangement results in a degree of redundancy to ensure that at least part of the treatment element is located at the region of co-aptation of the valve leaflets at all times.

In another arrangement, the treatment element may be located adjacent to a valve opening, externally of the valve opening and not extending through the valve opening.

The support element may be engageable with a wall of body tissue. The support element may be releasably engageable with a wall of body tissue. The support element may be configured to abut a wall of body tissue. The support element may be configured to exert a compressive force on a body tissue wall. The support element may be configured to abut an inner surface of a body tissue wall.

In one case the support element is configured to extend substantially laterally relative to a valve opening. The device may comprise a plurality of support elements connected together to form a substantially spherically-shaped support.

The support element may be engageable with a wall of an atrium of a heart. The support element may be engageable with at least one leaflet of a valve.

In one case the treatment element is carried on the support element. The support element may be substantially porous. The support element may be dimensioned, in use, to fit within a chamber of a heart. The support element may be substantially hollow and comprises a reticulated surface.

In one case the support element is substantially spherical, and the treatment element is provided on a portion of the spherical surface such that when the treatment element is positioned adjacent the interface between at least a pair of valve leaflets, the treatment element at least partially prevents leakage from said interface.

In another embodiment the support element comprises an anchor element to anchor the treatment element to a wall of body tissue. The anchor element may be extendable into a body tissue wall. The anchor element may be configured to extend only partially through a body tissue wall. The anchor element may be configured to be extended into a body tissue wall from an interior side of the body tissue wall.

In one case the anchor element comprises a hook element. The anchor element may comprise a suture loop. The anchor element may comprise a threaded element. The threaded element may comprise a screw element.

In one case the anchor element is configured to anchor the treatment element to a ventricle of a heart. The anchor element may be configured to anchor the treatment element to a septal wall of a ventricle of a heart. The anchor element may be configured to anchor the treatment element to the apex of a ventricle of a heart. The anchor element may be configured to anchor the treatment element to at least one leaflet of a valve.

In one case the support element comprises a connector element between the anchor element and the treatment element. The connector element may comprise a tether. The connector element may be configured to extend through a valve opening. By extending at least part of the support element through the valve opening, this arrangement may facilitate location of the treatment element at the region of co-aptation of the valve leaflets extending through the valve opening. The connector element may be dimensioned to extend, in use, from the anchor element through the interface between at least a pair of valve leaflets, to the treatment element.

The position at which the treatment element may be located along the connector element may be varied.

In one case the connector element comprises at least part of a guide wire, or a treatment wire. The connector element may have sufficient torsional rigidity to enable the connector element to be used to screw the anchor element to a wall of a heart.

In one embodiment the device comprises a delivery system to facilitate delivery of the treatment element to the region of co-aptation of the valve leaflets. The delivery system may comprise a percutatneous delivery system to facilitate percutaneous delivery of the treatment element to the region of co-aptation of the valve leaflets. The delivery system may comprise a delivery catheter for housing at least part of the treatment element during delivery. The delivery system may comprise a carrier element over which the treatment element is deliverable. The carrier element comprises a guidewire.

In one case the carrier element comprises an anchor element to anchor the carrier element to a wall of body tissue. The anchor element may be extendable into a body tissue wall. The anchor element may be configured to extend only partially through a body tissue wall. The anchor element may be configured to be extended into a body tissue wall from an interior side of the body tissue wall.

In one case the anchor element comprises a hook element. The anchor element may comprise a suture loop. The anchor element may comprise a threaded element. The threaded element may comprise a screw element.

In one case the anchor element is configured to anchor the carrier element to a ventricle of a heart. The anchor element may be configured to anchor the carrier element to a septal wall of a ventricle of a heart. The anchor element may be configured to anchor the carrier element to the apex of a ventricle of a heart.

In another case the delivery system comprises a holder element for holding the treatment element fixed relative to the carrier element. The holder element may comprise a clamp.

In one embodiment the treatment element is movable between a delivery configuration and a deployment configuration. The treatment element may be substantially collapsed in the delivery configuration. The treatment element may be substantially expanded in the deployment configuration. The treatment element may be biased towards the deployment configuration.

In one case the treatment element at least partially comprises a shape-memory material. The shape-memory material may comprise Nitinol.

In another case the treatment element is collapsible to facilitate delivery of the treatment element via a sheath or the like. The treatment element may be dimensioned when collapsed, to facilitate percutaneous delivery of the support element.

The treatment element of the medical device may be deployed using minimally invasive techniques. In particular it may be possible to deliver the treatment element to the region of co-aptation of the valve leaflets, and securely support the treatment element at the region of co-aptation using percutaneous techniques.

The treatment element of the medical device may be deployed using surgical techniques, for example using open heart surgery, and suturing the treatment element in position at the region of co-aptation of the valve leaflets.

In one embodiment the treatment element is at least partially comprised of a resiliently deformable material. The configuration of the treatment element may be adjustable in-situ at the region of co-aptation of the valve leaflets. The size of the treatment element may be adjustable in-situ. The radial dimension of the treatment element may be adjustable in-situ. The treatment element may be inflatable in-situ.

In one embodiment the treatment element comprises a non-thrombogenic coating. The coating may comprise polytetrafluoroethylene (PTFE).

The device may be configured for use in treatment of a heart valve. The device may be configured for use in treatment of an atrioventricular valve. The device may be configured for use in treatment of a mitral valve or a tricuspid valve. The treatment element may be configured to be located in an atrium of a heart. The treatment element may be configured to be located extending from an atrium of a heart at least partially through a mitral valve or a tricuspid valve.

In one case the radial dimension of the treatment element is substantially small relative to the overall radial dimension of a valve.

In one embodiment the device comprises a repair device for treating a leaking heart valve. The device may comprise a repair device for treating a leaking heart valve having at least a pair of valve leaflets.

In another aspect the invention provides a device for the treatment of a valve defect, the device comprising:
a treatment element; and
a treatment wire;
the treatment element having an expanded treatment configuration and a collapsed delivery configuration;
the treatment wire having a distal end, a proximal end, a distal segment, a transition segment, and a proximal segment;
the treatment wire comprising an anchor at the distal end;
the treatment element being slidable relative to the treatment wire and being lockable to the treatment wire.

In one embodiment of the invention the proximal segment of the treatment wire is detachable from the distal segment of the treatment wire. The proximal segment of the treatment wire may be configured to be located exterior to a patient. The transition segment may be adjacent a point of detachment of the proximal segment. The transition segment may be adapted to provide an atraumatic tissue implant interface. The atraumatic tissue implant interface may comprise a soft polymeric interface, or a porous interface, or a mechanical stress-distributing element.

The device may comprise a locking element for locking the treatment element to the treatment wire.

The invention also provides in a further aspect a device for the treatment of a valve defect, the device comprising:
a treatment element; and
a treatment wire;
the treatment element having an expanded treatment configuration and a collapsed delivery configuration;
the treatment wire having a distal end, a proximal end and a proximal segment;
the treatment wire comprising an anchor at the distal end;
the treatment element being connected to the treatment wire proximal of the distal end of the treatment wire.

In one case the treatment element is advanceable through a procedure catheter. The treatment element may be advanceable through a procedure catheter having a deflectable tip.

The treatment wire may be a wire, or a tube, or a combination of a wire and a tube. The treatment wire may be at least partially metallic or polymeric. The treatment wire may comprise an outer jacket and an inner core. The outer jacket may be polymeric and the inner core may be metallic. The core may be translatable or rotatable relative to the outer jacket. The inner core may be engagable with the anchor at the distal end of the treatment wire. Relative movement of the core may be configured to anchor the anchor in a wall of a heart. The core may be movable relative to the outer jacket to anchor the anchor in a wall of a heart. The inner core may be removable from the outer jacket. The outer jacket may be a non-thrombogenic polymer. The outer jacket may be coated or covered with a non-thrombogenic coating, and/or a drug eluting coating, and/or a coating containing an active agent, and/or an active agent and/or a drug.

In one case the treatment element is a self-expanding element. The treatment element may be expandable by inflation. The treatment wire may comprise a multi lumen tubing. At least one lumen may be an inflation lumen. The inflation lumen may be occludable after inflation. The inflation lumen may be occludable using a soft polymeric interface as a proximal plug or valve.

In another case the treatment element is expandable by mechanical actuation.

The core may be a pacing lead.

In one embodiment the treatment element is slidable over the treatment wire in the collapsed configuration and is coupled to the wire in the expanded configuration.

In a further aspect of the invention, there is provided a method of treating a valve, the method comprising the step of locating a treatment element at the region of co-aptation of leaflets of the valve to resist fluid flow in a first direction through an opening of the valve.

In one case the treatment element acts as a support element to at least partially support at least one of the valve leaflets at the region of co-aptation of the valve leaflets. The treatment element may act as an occluder element to at least partially occlude the valve opening.

In one embodiment the valve is a unidirectional valve. Fluid flow through the valve opening in a second direction may be facilitated. The second direction may be a forward direction. The first direction may be a retrograde direction.

In another case fluid flow through the valve opening urges the treatment element towards the valve opening. Fluid flow through the valve opening in the first direction may urge the treatment element towards the valve opening. Fluid flow through the valve opening in the second direction may urge the treatment element towards the valve opening.

In one case the treatment element is engaged with at least one leaflet of the valve. The valve leaflet may be movable between a closed configuration and an open configuration. In the closed configuration the treatment element may engage with the valve leaflet. In the closed configuration the treatment element may sealingly engage with the valve leaflet. In the closed configuration the treatment element may prevent fluid flow through the valve opening. In the open configuration the treatment element may be spaced-apart from the region of co-aptation of the valve leaflets. In the open configuration the treatment may resist fluid flow in the first direction through the valve opening. In the open configuration the treatment element may facilitate fluid flow in the second direction through the valve opening.

In another embodiment the treatment element engages the valve leaflet at an engagement region spaced substantially from an annulus of the valve. The treatment element may engage the valve leaflet at the region of co-aptation of the valve leaflets. The treatment element may engage the valve leaflet at an engagement region in proximity to or within the valve opening.

The treatment element may be inserted into a position adjacent an interface of the valve leaflets such that the treatment element at least partially prevents leakage from said interface. The method may comprise, in the step of inserting the treatment element, percutaneously inserting the treatment element.

In another embodiment the method comprises the step of supporting the treatment at the region of co-aptation of the valve leaflets. The treatment element may be supported adjacent to the valve opening. The treatment element may be supported externally of the valve opening. The treatment element may be supported extending at least partially through the valve opening.

In one case the treatment element is supported using a support element. The method may comprise the step of engaging the support element with a wall of body tissue. The support element may abut the body tissue wall. The support element may exert a compressive force on the body tissue wall. The support element may abut an inner surface of the body tissue wall. The support element may engage a wall of an atrium of a heart. The support element may engage at least one leaflet of the valve.

In another embodiment the treatment element is anchored to the body tissue wall. The treatment element may be anchored to the body tissue wall from an interior side of the body tissue wall. The treatment element may be anchored to a ventricle of a heart. The treatment element may be anchored to a septal wall of the heart ventricle. The treatment element may be anchored to the apex of the heart ventricle. The treatment element may be anchored to at least one leaflet of the valve.

In one case at least part of the support element is extended through the valve opening.

The method may comprise the step of deploying the support element to secure the treatment element in position.

The method may comprise the step of tethering the treatment element via the support element, to a wall of a heart, preferably a wall of a ventricle of the heart.

In one case the method comprises the step of providing the treatment element on the support element, the support element being porous, and lodging the support element within the atrium such that the treatment element is located adjacent the interface of the valve leaflets in order to at least partially prevent leakage from said interface.

In another case the method comprises the step of delivering the treatment element to the region of co-aptation of the valve leaflets. The treatment element may be percutaneously delivered.

In one case the method comprises the step of housing at least part of the treatment element in a delivery catheter before delivering the treatment element. The treatment element may be delivered over a carrier element. The method may comprise the step of locating the carrier element in a desired location relative to the valve before delivering the treatment element over the carrier element. The carrier element may be located extending through the valve opening. The method may comprise the step of anchoring the carrier element to a wall of body tissue. The carrier element may be anchored to the body tissue wall from an interior side of the body tissue wall. The carrier element may be anchored to a ventricle of a heart. The carrier element may be anchored to a septal wall of the heart ventricle. The carrier element may be anchored to the apex of the heart ventricle.

In a further case the method comprises the step of, after delivering the treatment element over the carrier element, holding the treatment element fixed relative to the carrier element. The treatment element may move from a delivery configuration to a deployment configuration. The method may comprise the step of collapsing the treatment element, percutaneously passing the treatment element into the atrium, and expanding the treatment element. In one case the method comprises the step of performing imaging to assist in locating the treatment element at the region of co-aptation of the valve leaflets. X-ray and/or ultrasound imaging may be performed.

In one case the invention provides a method of treating a heart valve. In another case the invention provides a method of treating an atrioventricular valve. In a further case the invention provides a method of treating a mitral valve or a tricuspid valve.

The method may comprise the step of locating the treatment element in an atrium of a heart. The method may comprise the step of locating the treatment element extending from an atrium of a heart at least partially through a mitral valve, or a tricuspid valve.

In another case the invention provides a method of treating a leaking human or animal heart valve having at least a pair of valve leaflets.

The invention also provides in another aspect a method of treating a valve using a treatment device, the treatment device comprising a treatment element, a treatment wire and an anchor element, the method comprising the steps of:
advancing a procedural catheter into the atrium;
advancing the treatment wire through the procedural catheter and passing the distal end of the treatment wire across the valve;
anchoring a distal end of the treatment wire to a wall of the ventricle;
expanding the treatment element at the desired region; and
terminating the proximal end of the wire beneath the skin of the patient.

In one embodiment the method comprises the step of steering the procedural catheter to allow ease of advancement of the treatment device. The steering step may comprise torqueing a shaped procedural catheter. The steering step may comprise actuating a pull cable to deflect a soft distal segment of the procedural catheter.

In one case the method comprises the step of collapsing the treatment element.

In one embodiment the anchoring step comprises a relative motion between a core of the treatment wire and an outer tube of the treatment wire. The anchoring step relative motion may comprise torqueing the core relative to the outer tube to anchor a distal end of the treatment wire. The anchoring step relative motion may comprise advancing the core relative to the outer tube to anchor a distal end of the treatment device.

In one embodiment the method comprises the step of inserting the collapsed treatment element into the procedural catheter. The method may comprise the step of advancing the treatment element over the treatment wire. The step of terminating the proximal end of the treatment wire may comprise the step of removing a proximal segment of the wire. The step of removing a proximal end of the treatment wire may comprise cutting, and/or unscrewing, and/or decoupling, and/or cutting, and/or breaking the proximal end of the wire. The step of terminating the proximal end of the treatment wire may comprise engaging a soft cap with the end of the wire. The step of terminating the proximal end of the treatment wire may comprise closing the puncture site with the proximal end of the treatment wire beneath the skin.

In one case the method comprises the step of locking the treatment element to the treatment wire adjacent the valve. The step of collapsing the treatment element may comprise the step of loading the treatment element into a delivery catheter distal end.

In one embodiment the method comprises the step of advancing the treatment element and the delivery catheter over the treatment wire. The method of advancing the catheter comprises a rapid exchange technique.

In one case the step of expanding the treatment element comprises retraction of the sheath relative to a fixing abutment. The method may comprise the step of adjusting the position of the treatment device relative to the valve. The position adjusting step may comprise visualising the treatment device under fluoroscopy using radiopaque markers on the treatment device. The position adjusting step may comprise visualising the treatment device using an ultrasound probe and ultrasound visible markers positioned on the treatment device.

In another aspect of the invention, there is provided a method of delivering a medical device to a desired location in a heart, the method comprising the step of advancing the medical device through a coronary sinus to the desired location.

In one embodiment the method comprises the step of advancing the medical device out of the coronary sinus to the desired location. The method may comprise the step of forming a first opening in the sidewall of the coronary sinus. The medical device may be advanced out of the coronary sinus through the first opening. The medical device may be drawn out of the coronary sinus.

In one case the medical device is advanced over a carrier element. The carrier element may be advanced through the coronary sinus. The carrier element may be advanced out of the coronary sinus. The carrier element may be advanced out of the coronary sinus through the first opening. The carrier element may be drawn out of the coronary sinus.

In another embodiment the method comprises the step of advancing a drawing element, for drawing the medical device and/or the carrier element out of the coronary sinus, through the coronary sinus. The method may comprise the step of advancing the drawing element out of the coronary sinus. The method may comprise the step of forming a second opening in the sidewall of the coronary sinus. The drawing element may be advanced out of the coronary sinus through the second opening. The medical device and/or the carrier element may be drawn towards the second opening.

In one case the method comprises the step of supporting the medical device in the desired location.

The medical device may be advanced to the desired location at the region of co-aptation of valve leaflets of the heart. The medical device may be advanced to the desired location at the region of co-aptation of mitral valve leaflets or tricuspid valve leaflets of the heart.

In one case the invention provides a method of delivering a treatment element.

The invention provides in another aspect a method of treating a valve, the method comprising the step of delivering a medical device to a desired location in a heart as described above.

In a further aspect of the invention there is provided a delivery catheter for delivering at least one medical device through a coronary sinus, the catheter comprising at least one opening through which at least one medical device is advanceable out of the catheter through a sidewall of the coronary sinus.

In one embodiment of the invention the catheter comprises a first opening through which a first medical device is advanceable out of the catheter and a second opening through which a second medical device is advanceable out of the catheter. The opening may be provided in sidewall of the catheter. The catheter may comprise an opening forming element for forming an opening in a sidewall of the coronary sinus. The catheter may comprise at least one drawing element for drawing at least one medical device out of the coronary sinus.

The present invention provides, in one aspect, a repair device for treating a leaking heart valve having at least a pair of valve leaflets, the repair device comprising a plug for location adjacent an interface of the leaflets such that the plug at least partially prevents leakage from said interface; and a support adapted to secure the plug in said position.

Preferably, the plug is collapsible in order to facilitate the delivery of the plug via a sheath or the like.

Preferably, the plug is dimensioned when collapsed, to facilitate the percuatneous delivery of the support.

Preferably, the plug, when deployed, is shaped and dimesioned to permit the unidirectional flow of fluid therepast.

Preferably, the repair device comprises a guide wire for delivering the plug and the support.

Preferably, the plug is at least partially comprised of a resiliently deformable material.

Preferably, the plug is at least partially comprised of a non thrombogenic material.

Preferably, the plug has a substantially fluid impermeable contact surface for location adjacent the interface of the leaflets such that the plug at least partially prevents leakage from said interface.

Preferably, the contact surface is substantially circular, conical, or cylindrical.

Preferably, the support comprises an anchor; and a tether secured between the anchor and the plug. Preferably, the anchor comprises a screw adapted to be anchored to a wall of the heart. Preferably, the tether is dimensioned to extend, in use, from the anchor, through the interface of the leaflets, to the support. Preferably, the position at which the plug is located along the tether may be varied. Preferably, the support is secured to a leading end of the guide wire. Preferably, the guide wire has sufficient torsional rigidity to enable the guide wire to be used to screw the anchor to the wall of the heart.

Alternatively, the plug is carried on the support, the support being porous and being dimensioned, in use, to fit within a chamber of the heart. Preferably, the support is hollow and comprises a reticulated surface. Preferably, the support is substantially spherical, and the plug is provided on a portion of the spherical surface such that when the plug is positioned adjacent the interface of the leaflets, the plug at least partially prevents leakage from said interface.

According to another aspect of the present invention there is provided a method of treating a leaking human or animal heart valve having at least a pair of valve leaflets, the method comprising the steps of inserting a plug into a position adjacent an interface of the leaflets such that the plug at least partially prevents leakage from said interface; and securing the plug in said position.

Preferably, the method comprises, in the step of inserting the plug, percutaneously inserting the plug.

Preferably, the method comprises, in the step of securing the plug, deploying a support to secure the plug in position.

Preferably, the method comprises, in the step of securing the plug, tethering the plug, via the support, to a wall of the heart, preferably a wall of a ventricle of the heart.

Alternatively, the method comprises, in the step of securing the plug, providing the plug on the support, the support being porous, and lodging the support within the atrium such that the plug is located adjacent the interface of the leaflets in order to at least partially prevent leakage from said interface.

Preferably, the method comprises in the step of inserting the plug, collapsing the plug, percutaneously passing the plug into the atrium, and expanding the plug.

In one case the invention provides a percutaneous cardiac valve repair device and method. The method may include the step of introducing a support structure through a vein in the neck to buttress the mitral valve or tricuspid valve. The treatment element of the medical device may be delivered percutaneously with a procedure similar to cardiac catheterization. The treatment element of the medical device may be manufactured out of nitinol, “memory metal”, metal that can be compressed into small tubes but will return to its original shape once delivered from the tube. In the medical device, the support for the treatment element may be provided by a sheath and a wire attached to the apex of the heart ventricle. The support may be provided by the treatment element engaging the walls of the atrial cavity. The shape, size and position of the medical device may be altered to achieve the desired result. The treatment element of the medical device may be removable and the position of the treatment element may be altered at a later date by movement of the locking system in the neck The design of the treatment element of the medical device is such that it may also form part of the valvular surface in conditions of severe regurgitation.

The carrier element/support wire may be inserted through the venous system, through the inferior vena cava or the superior vena cava to the right atrium or across the atrial septum into the left atrium.

The soft support treatment element may be delivered over the wire. The position and size of which can be varied. The treatment element prevents prolapse of leaflets, aiding apposition and plugging the defect in the valve.

The nitinol valve leaflet umbrella-like support treatment element prevents prolapse of the valve leaflets. Its position and size may be varied depending on the amount it is advanced out of the sheath.

The location of the treatment element is important for its performance. The treatment element should sit at, or close to, the level of valve co-aptation (closure). This position will vary between individuals and at different times during the cardiac cycle of contraction and relaxation. It may also be affected by posture and respiration.

The treatment element may be delivered over a support wire which crosses the regurgitant valve. The position of the treatment element may be varied along this support wire to ensure the correct location is achieved. In addition the active surface of the treatment element may be relatively long to allow an amount of redundancy in device positioning. The device may be delivered using x-ray and ultrasound imaging to ensure its correct location.

The stability of the treatment element within the heart is important for its performance. There are a number of forces that act on the treatment element; 1) regurgitant flow from the ventricle into the atrium 2) forward flow from the body into the ventricle when the valve is open and 3) gravity and other minor forces such as respiration and body movement.

The stability of the treatment element may be maintained either by a wire support anchored in the ventricular wall, or by supports anchored in the walls of the atrium. The shape of the treatment element may be designed to use the regurgitant jet to force it into the correct position.

The covering and/or surface of the medical device may be configured to limit the possiblity of thrombosis. A polytetrafluoroethylene (PTFE) covering may be employed.

As used in this patent specification, the term “interface” will be understood to mean an area at which two elements or surfaces meet or approach one another without necessarily touching.

As used in this patent specification, the term “plug” will be understood to mean a component or collection of components which are adapted to at least partially fill or occlude a gap between two or more surfaces or the like, whether using the whole plug or a portion thereof.

As used in this patent specification, the term “repair” will be understood to mean the procedure of resisting retrograde fluid flow through a valve, for example by at least partially supporting at least one of the valve leaflets at the region of co-aptation of the valve leaflets and/or by at least partially occluding the valve opening.

### BRIEF DESCRIPTION OF THE DRAWINGS

The invention will be more clearly understood from the following description of some embodiments thereof, given by way of example only, with reference to the accompanying drawings, in which:

FIG. 1 is a schematic illustration of a heart;

FIG. 2 illustrates a schematic representation of a first embodiment of a medical device suitable for use in treatment of a valve according to the invention, deployed in situ in a human heart;

FIGS. 3 and 4 are cross-sectional, side views of a delivery system of the device of FIG. 2;

FIG. 5 illustrates a first stage in the insertion of the device of FIG. 2;

FIG. 6 illustrates the following stage in the insertion of the device of FIG. 2;

FIG. 7 illustrates a further stage in the insertion of the device of FIG. 2, where a treatment element of the device is being deployed from a sheath of the delivery system of FIGS. 3 and 4 utilised to deliver the treatment element to the heart;

FIG. 8 illustrates the treatment element when deployed in situ, with the delivery sheath still in position;

FIG. 9 illustrates the treatment element when deployed in situ having been clamped in the correct position, and the delivery sheath removed;

FIG. 10 is an isometric view of a treatment element of another medical device according to the invention;

FIG. 11 is an end view of the treatment element of FIG. 10;

FIG. 12 is an end view of the treatment element of FIG. 10, in use;

FIGS. 13 to 15 are views similar to FIGS. 10 to 12 of a treatment element of another medical device according to the invention;

FIGS. 16 to 18 are views similar to FIGS. 10 and 12 of a treatment element of a further medical device according to the invention;

FIG. 19 is a cut-away, isometric view of another medical device according to the invention, in use;

FIG. 20 is a view similar to FIG. 19 of a further medical device according to the invention, in use;

FIG. 21 is a side view of another medical device according to the invention, in use;

FIGS. 22 to 26 are side views of further medical devices according to the invention, in use;

FIG. 27 is an isometric view of another medical device according to the invention;

FIG. 28 is an end view of the device of FIG. 27;

FIG. 29 is an isometric view of another medical device according to the invention, in use;

FIG. 30 is an end view of the device of FIG. 29;

FIG. 31 illustrates a schematic representation of another embodiment of a medical device according to the invention, deployed in a final or working configuration in a human heart;

FIG. 32 is an isometric view of another medical device according to the invention, in use;

FIG. 33 is a cross-sectional, side view of a further medical device according to the invention, in use;

FIGS. 34 and 35 are side views of a support element of another medical device according to the invention, in use;

FIGS. 36 to 38 are side views of support elements of further medical devices according to the invention;

FIGS. 39 to 52 are cross-sectional, side views of another medical device according to the invention, in use;

FIG. 53 to 63 are cross-sectional, side views of a further medical device according to the invention, in use;

FIG. 64 is an isometric view of another medical device according to the invention, in use;

FIG. 65 is an end view of the device of FIG. 64;

FIG. 66 is an isometric view from the side of another medical device according to the invention;

FIG. 67 is a side view of the device of FIG. 66;

FIG. 68 is an isometric view from an end of the device of FIG. 66;

FIG. 69 is an end view of the device of FIG. 66;

FIG. 70 illustrates a schematic representation of the first stage of insertion of another embodiment of a medical device according to the invention;

FIG. 71 illustrates the following stage in the insertion of the device of FIG. 70, in which a treatment element of the device is being deployed from a delivery sheath of the device;

FIG. 72 illustrates a further stage in the insertion of the device of FIG. 70;

FIG. 73 illustrates the treatment element of the device of FIG. 70 when fully deployed within an atrium of a human heart, with a guide wire of the device remaining in position within the heart;

FIG. 74 illustrates the fully deployed treatment element of the device of FIG. 70, when the guide wire of the device has been removed from the heart; and

FIGS. 75 to 80 are cut-away, isometric views of another medical device according to the invention, in use.

### DETAILED DESCRIPTION

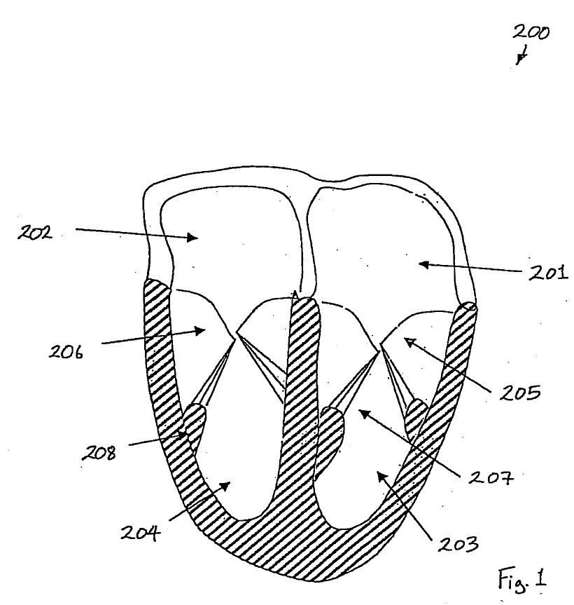

FIG. 1 illustrates the anatomy of a heart 200. The heart 200 has a left atrium 201, a right atrium 202, a left ventricle 203 and a right ventricle 204. Also illustrated are the mitral valve 205, the tricuspid valve 206, the chordae tendiniae 207 and the papillary muscle 208.

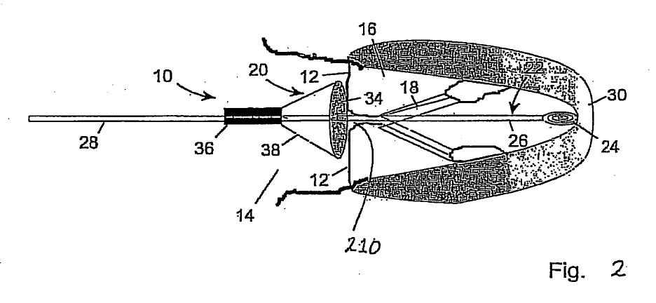

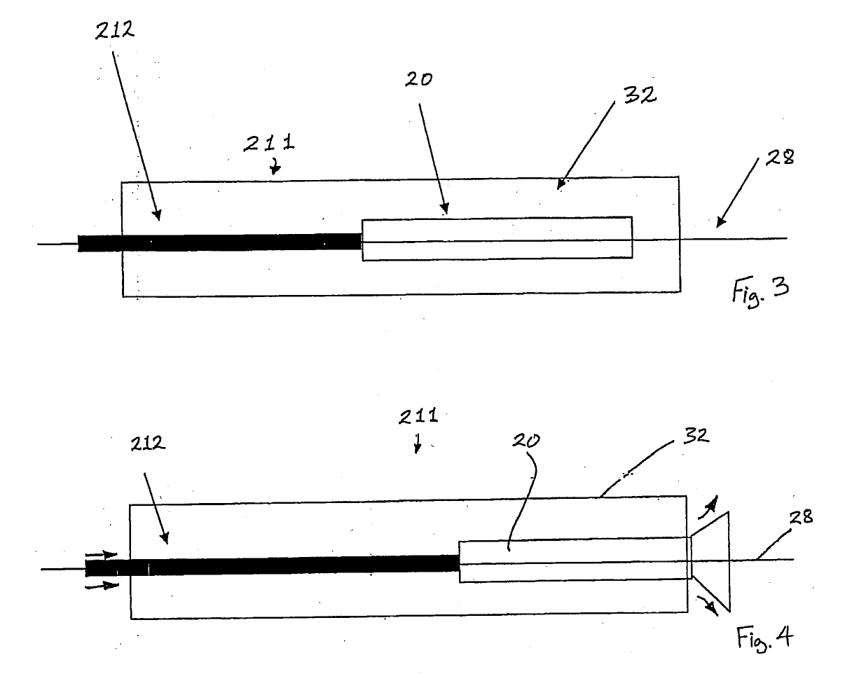

Referring to FIGS. 2 to 9 there is illustrated a medical device 10 according to the invention. The device 10 is suitable for use in treatment of a valve. The device 10 is particularly suitable for treating the mitral valve 205 to prevent retrograde blood flow through the mitral valve 205.

Referring to FIGS. 2 to 9, there is illustrated the medical device 10 which acts as a repair device, for treating leaking of the heart valve leaflets 12, in particular the atrioventricular valve leaflets 12, in order to substantially reduce or eliminate regurgitation of blood through the valve leaflets 12. Although throughout the following description explicit reference is made to the valve leaflets 12 located between an atrium 14 and a ventricle 16 of a heart, it is to be appreciated that the device 10 of the present invention, in addition to the surgical method of the invention as hereinafter described, are applicable to other valves within the heart.

The valve leaflets 12 are capable of deforming inwardly from the position shown in FIG. 2 into the ventricle 16, in order to allow blood to be pumped from the atrium 14 into the ventricle 16, from where it is then pumped to the lungs or body, depending on whether the ventricle 16 is the left ventricle or the right ventricle. The valve leaflets 12 are prevented from opening outwardly into the atrium 14 by a pair of cordae tendinae 18, each of which is connected between the ventricle 16 and the respective valve leaflet 12. The pair of valve leaflets 12, when functioning correctly, therefore act as one way valve or gate which ensures that blood flows through the heart in the correct direction, namely from the atrium 14 to the ventricle 16. However, the valve leaflets 12 do not always work as they should, mainly because of problems caused by disease, age, or a congenital defect. One of the main problems affecting the valve leaflets 12 arises when the valve leaflets 12 fail to correctly or completely align, which can allow blood to flow back from the ventricle 16 into the atrium 14, known as regurgitation.

The medical device 10 comprises a treatment element 20 which is configured to be located at the region of co-aptation of the mitral valve leaflets 12 to resist fluid flow in the retrograde direction through the valve opening 210, a support element 22 to support the treatment element 20 at the region of co-aptation of the valve leaflets 12, and a delivery system 211 to facilitate delivery of the treatment element 20 to the region of co-aptation of the valve leaflets 12. In particular the medical device 10 resists fluid flow in the retrograde direction through the valve opening 210 by at least partially supporting at least one of the valve leaflets 12 at the region of co-aptation of the valve leaflets 12, and/or by at least partially occluding the valve opening 210.

Referring in particular to FIG. 2, the treatment element 20 is provided in the form of a plug 20 which is adapted, as will be described in greater detail hereinafter, to be located adjacent the interface of the leaflets 12 such that the plug 20 at least partially prevents leakage from said interface, by partially or completely occluding said interface, and therefore prevent regurgitation of blood therefrom. In order to secure the plug 20 in position, the device 10 is provided with the support element 22 which, in the embodiment illustrated, comprises an anchor 24 which is secured, as will be described in detail hereinafter, to the septal wall of the ventricle 16 or to the apex of the ventricle 16, the support element 22 further comprising a tether 26 extending in use from the anchor 24, between the valve leaflets 12, into connection with the plug 20. The support 22 therefore retains the plug 20 in position relative to the leaflets 12, such that each time the valve leaflets 12 close, the plug 20 will at least partially occlude any gap therebetween.

The plug 20 preferably comprises a substantially fluid impermeable contact surface 34 which is disposed, in use, against or between the gap or interface between the valve leaflets 12, the plug 20 also comprising a base 36 which is connected to the contact surface 34 via a plurality of connecting struts 38. The contact surface 34 and the struts 38 are preferably formed from a resiliently deformable material such as nitinol metal or the like, in order to allow the plug 20 to be displaced into a collapsed state and to self-expand to an expanded state. The plug 20 is also preferably formed from a non-thrombogenic material.

The delivery system 211, illustrated in FIGS. 3 and 4, comprises a delivery catheter sheath 32 for housing at least part of the treatment element 20 during delivery, and a carrier element 28 over which the treatment element 20 is delivered.

The treatment element 20 is movable between a collapsed, delivery configuration (FIG. 3) and an expanded, deployment configuration (FIG. 4). During delivery, the treatment element 20 is housed in the delivery catheter sheath 32 in the collapsed, delivery configuration (FIG. 3), and upon deployment the treatment element 20 expands to the expanded, deployment configuration upon release from the delivery catheter 32 (FIG. 4).

A delivery wire 212 may be advanced to deliver the treatment element 20 from the sheath 32 (FIG. 4). As the treatment element 20 exits the delivery sheath 32 it re-expands to its natural conformation.

In this case the carrier element 28 is provided in the form of a support guide wire 28.

It will be appreciated that the device 10 may be located within the heart by utilising conventional open heart surgery. However a significant benefit of the device 10 is that it can be located in place by using minimally invasive surgical techniques. Thus the preferred method of insertion of the device 10 consists of the percutaneous insertion of the guide wire 28 into the atrium 14, between the valve leaflets 12, and into the ventricle 16. A portion of the guide wire 28 will act as the tether 26.

In use, the guide wire 28 is inserted percutaneously through a vein in the neck or groin, in similar fashion to the well established process for the insertion of a pacemaker. Located at the end of the guide wire 28 is the anchor 24, which in the embodiment illustrated is in the form of a self tapping screw element. It will be appreciated that the anchor 24 could be of any other suitable form, for example being provided with self retaining tines or barbs or the like. The guide wire 28, and in particular the anchor 24, is advanced through the ventricle 16 until a wall, preferably the septal wall 30 thereof, is contacted by the anchor 24. This process is preferably aided by the use of echocardiographic and x-ray imaging equipment or the like. Once the anchor 24 has contacted the septal wall 30, the guide wire 28 is rotated about a longitudinal axis thereof (FIG. 5), to rotate the anchor 24, thus threading the anchor 24 into the septal wall 30 in order to effect a robust connection between the guide wire 28 and the ventricle 16.

The sheath of the delivery catheter 32 is then delivered over the guide wire 28, until a free end of the catheter 32 is in communication with the atrium 14. At this point, and referring to FIGS. 6 and 7, the plug 20 is advanced through and out of the catheter 32, towards the valve leaflets 12. Although the plug 20 is illustrated as being dimensioned to fit within the catheter 32, this is for illustrative purposes only, and in general the plug 20 will be significantly larger in diameter than the catheter 32. For this reason the plug 20 is preferably resiliently deformable such as to be displaceable between the collapsed state (FIG. 3) and the expanded state (FIG. 8). The plug 20 can therefore initially be inserted into the catheter 32 in the collapsed state, advanced out of the catheter 32, and on exiting the catheter 32 into the atrium 14 will automatically assume the expanded state, as illustrated in FIG. 8.

Referring to FIG. 8, the plug 20 is advanced along the guide wire 28 until the contact surface 34 is correctly positioned against the valve leaflets 12. The base 36 may then be clamped against the guide wire 28, by the release of a remotely operable spring loaded clamp or the like contained within the base 36, or on the guide wire 28, at which point the plug 20 is secured against the valve leaflets 12 by means of the tether 26 connected between the septal wall 30 and the plug 20. FIG. 8 illustrates the plug 20 positioned at the desired level within the atrium 14 supporting the valve leaflets 12 and plugging the defect.

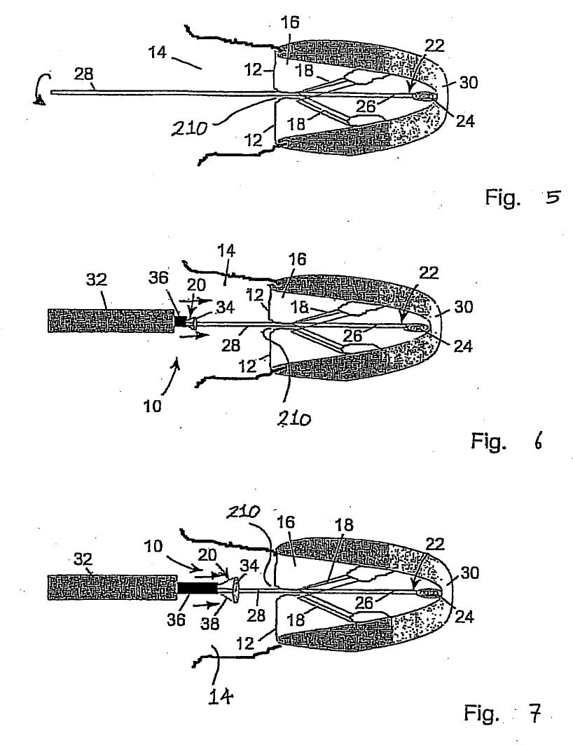

Referring to FIG. 9, the sheath 32 is then removed back along the guide wire 28 and out of the patient's vein, leaving only the guide wire 28 in position. The opposed end of the guide wire 28, at the point of incision into the patient, may be provided with any suitable subcutaneous plug or the like in order to secure the guide wire 28 in position. The device 10 is thus secured in place and ready for use, with the plug 20, and in particular the contact surface 34, allowing blood to flow therepast from the atrium 14 into the ventricle 16, while at least partially preventing the regurgitation of blood by occluding the gap at the interface of the valve leaflets 12. It should therefore be appreciated that the diameter of at least the contact surface 34 should be sufficiently large to substantially occlude any such gap to the extent that backward leakage is reduced by an effective amount, and preferably entirely, while being sufficiently small to allow the flow of blood around the contact surface 34 and into the ventricle 16.

It will be appreciated that the configuration and/or shape of the treatment element 20 may be varied to suit the requirements and characteristics of a particular patient anatomy.

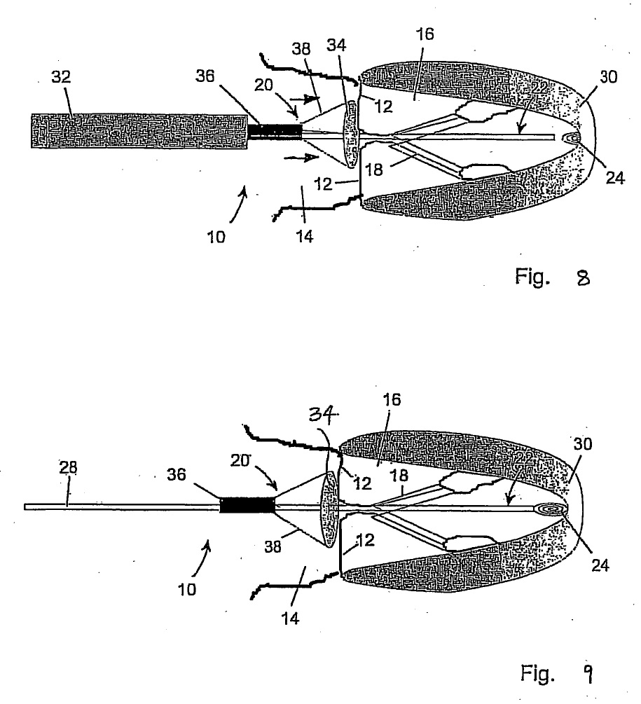

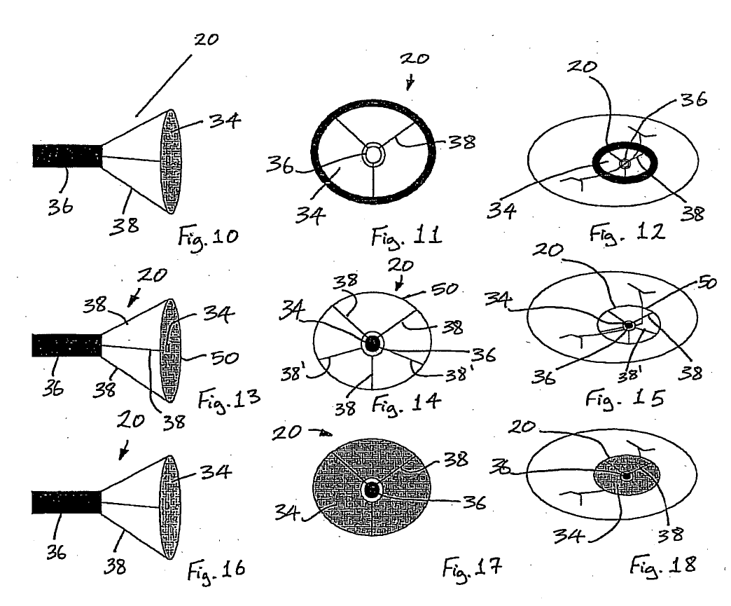

For example, FIGS. 10 to 12, 13 to 15 and 16 to 18 illustrate three alternative configurations for the treatment element 20.

Referring to FIG. 11, there is illustrated a front elevation of an alternative embodiment of the plug 20, in which like components have been accorded like reference numerals. The plug 20 comprises the base 26 extending from which are three struts 38. Mounted to the struts 38 is a ring shaped contact surface 34 which, in use, will be seated against the valve leaflets 12 in order to substantially occlude any gap therebetween. The ring shaped contact surface 34 could be used when only a small gap exists between the valve leaflets 12, and presents a significantly smaller impediment to the flow of blood from the atrium 14 into the ventricle 16.

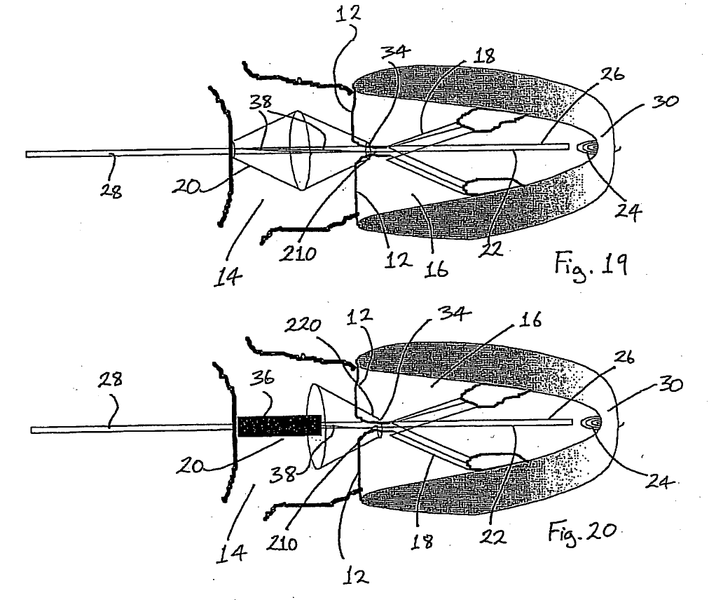

Referring to FIG. 14, another embodiment of the plug 20 is illustrated. The plug 20 compries the base 36 extending from which are three struts 38, which connect to a support ring 50. Extending radially inwardly from the support ring 50 are a pair of secondary struts 38′, which carry the contact surface 34 at the centre of the support ring 50. In use, the plug 20 is positioned such that the contact surface 34 at least partially occludes any gap between the valve leaflets 12, as hereinbefore described. The contact surface 34 is significantly smaller than the contact surface 34 of the plug 20 of the FIGS. 2 to 9, and would thus be used when a small gap exists between the valve leaflets 12. The contact surface 34 will present a significantly smaller impediment to the flow of blood from the atrium 14 into the ventricle 16.

As illustrated in FIGS. 17 and 18 in particular, the contact surface 34 of the treatment element 20 may be formed of a membranous or plastic material to fill the valve defect.

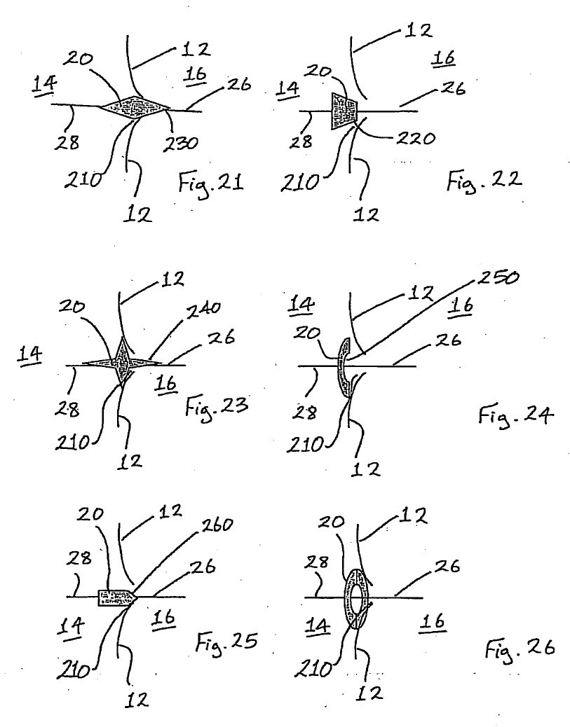

FIGS. 19 and 20 illustrate two further alternative configurations for the treatment element 20. For example the treatment element 20 of FIG. 19 has a substantially diamond shape. For example the treatment element 20 of FIG. 20 has a substantially frusto-conical shape with the apex 220 of the cone pointing in the direction of the valve opening 210. The cone of the treatment element 20 extends partially through the valve opening 210, in this case.

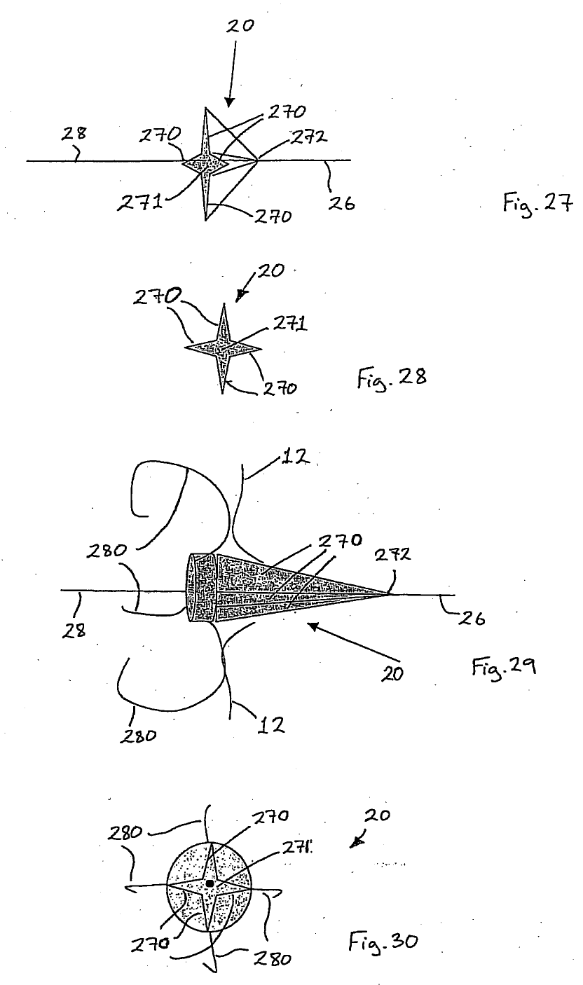

In FIGS. 21 to 26 there are illustrated six other alternative configurations for the treatment element 20. For example, the treatment element 20 of FIG. 21 has a substantially diamond shape with an apex 230 of the diamond pointing towards the valve opening 210 and extending into the valve opening 210, the treatment element 20 of FIG. 22 has a substantially frusto-conical shape with the apex 220 of the cone pointing towards the valve opening 210, the treatment element 20 of FIG. 23 has the shape of a four-armed star with one arm 240 of the star pointing towards the valve opening 210 and extending into the valve opening 210, the treatment element 20 of FIG. 24 has a substantially crescent shape with the concave portion 250 of the crescent facing towards the valve opening 210, the treatment element 20 of FIG. 25 has a pointed tip 260 at one end of the treatment element 20 with the tip 260 pointing towards the valve opening 210 and extending into the valve opening 210, the treatment element 210 of FIG. 26 has a substantially oval or elliptical shape with the major axis of the ellipse substantially perpendicular to the guide wire 28 and the tether 26 and the minor axis of the ellipse substantially parallel to the guide wire 28 and the tether 26.

Referring to FIGS. 27 to 30 there are illustrated two further alternative configurations for the treatment element 20.

For example the treatment element 20 of FIGS. 27 and 28 has four fin arms 270 which extend radially outwardly from a central body portion 271. Each arm 270 tapers inwardly to a point as the arm 270 extends away from the body portion 271, as illustrated in FIG. 28. The treatment element 20 thus has a shape similar to a four-armed star. The fins 270 act to direct the treatment element 20 towards the regurgitant orifice 210.

The treatment element 20 tapers inwardly to a point 272 in the longitudinal direction parallel to the guide wire 28 and the tether 26. In use, the point 272 extends into the valve opening 210.

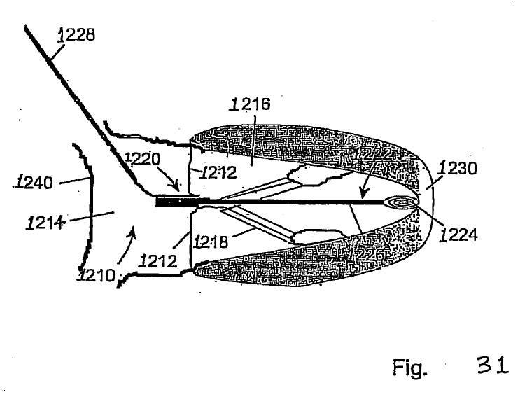

For example, in the medical device of FIGS. 29 and 30 four support arms 280 extend radially outwardly from the body portion 271 of the treatment element 20. The arms 280 are engageable with the inner walls of the atrium and with the valve leaflets 12 to support the treatment element 20 in the desired location at the region of co-aptation of the valve leaflets 20 with the treatment element 20 extending partially into the valve opening 210. In this case the arms 280 are curved for atrial support.

Referring to FIG. 31 there is illustrated another medical device according to the invention, generally indicated as 1210, which is similar to the medical device 10 of FIGS. 2 to 9. The device 1210 comprises a generally cylindrical plug 1220 for location between a pair of valve leaflets 1212 situated between an atrium 1214 and a ventricle 1216 of a heart. The leaflets 1212 are connected to the ventricle 1216 by a respective set of cordae tendinae 1218.

The device 1210 comprises a support 1222 having an anchor 1224 and a tether 1226, the tether 1226 being provided at the end of the guide wire 1228 which is initially utilised in the insertion of the plug 1220 in a manner similar to that as hereinbefore described with reference to FIGS. 2 to 9. The anchor 1224 is secured, in use, to a septal wall 1230, while the guide wire 1228 exits the atrium 1214 through a vein adjacent a rear wall 1240 thereof.

A difference between the device 1210 of FIG. 31 and the device 10 of FIGS. 2 to 9 is the use of a cylindrical plug 1220, which may have any suitable cross-sectional shape, to occlude the gap between the leaflets 1212. The plug 1220 preferably includes a remotely actuatable clamp therein, as described with reference to the base 36 of the device 10 of FIGS. 2 to 9, in order to allow the plug 1220 to be secured to the guide wire 1228 or the tether 1226. The device 1210 operates in a manner similar to the device 10 of FIGS. 2 to 9.

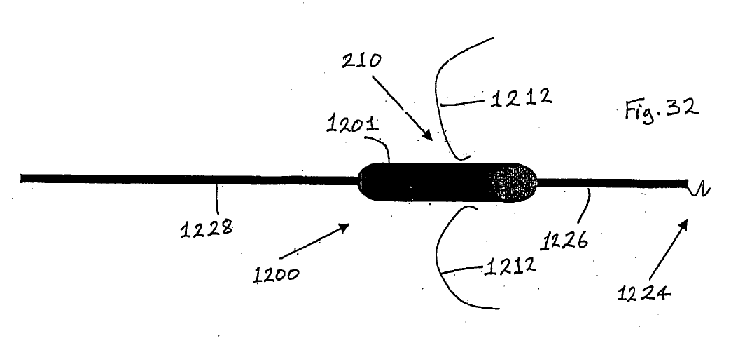

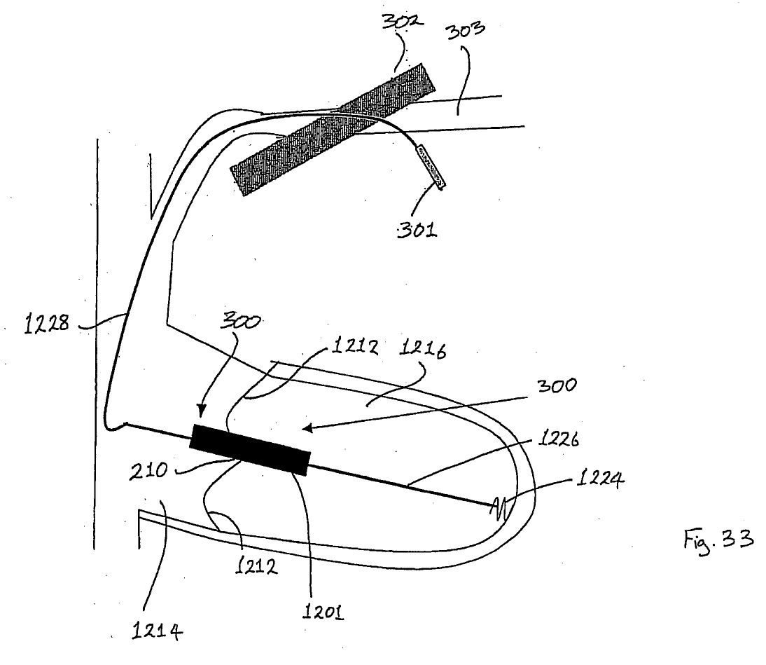

FIG. 32 illustrates another medical device 1200 according to the invention, which is similar to the device 1210 of FIG. 31, and similar elements in FIG. 32 are assigned the same reference numerals.

The lead/support wire 1226 is fixed in the ventricular muscle using the anchor element 1224.

The profile of the treatment element 1201 ensures that fluid flow impinging on the treatment element 1201 directs the treatment element 1201 into the correct position at the region of co-aptation of the valve leaflets 1212 extending through the valve opening 210.

In use, the valve leaflets 1212 co-apt against the expansion 1201 on the lead 1228. The expansion 1201 can be many shapes and lengths. The width or radial dimension of the expansion 1201 can be varied either by delivering different sized treatment elements or by inflating or deflating its elastic wall.

It will be appreciated that the guide wire lead 1228, the treatment element 1201 and the tether 1226 may be integrally formed. In this case the treatment element 1201 may be formed as an expansion section on the lead 1228, which may be anchored to the ventricle wall be means of the anchor element 1224. This results in a particularly simple form of the medical device 1200.

The guide wire lead 1228 and the tether 1226 may be integrally formed from a single wire, for example a single pacing lead.

The treatment element 1201 may be self-actuating. The treatment element 1201 may be actuated by the action of withdrawing a retaining sheath. The treatment element 1201 may be at least partially of a shape-memory material, such as Nitinol, to assist in actuating the treatment element 1201.

The treatment element 1201 may be formed in any one of a number of possible shapes and configurations. For example the treatment element 1201 may have a semi-lunar shape which may be suitable for use with a mitral valve which has a semi-lunar shaped opening.

In FIG. 33 there is illustrated a further medical device 300 according to the invention, which is similar to the device 1200 of FIG. 32, and similar elements in FIG. 33 are assigned the same reference numerals.

The treatment element plug 1201 extends through the valve opening 210 in this case.

In this case the guide wire 1228 is illustrated extending from the heart proximally through the subclavian vein 303 passed the clavicle bone 302 of the patient.

A proximal end 301 of the guide wire lead 1228 may be sutured to muscle tissue beneath the outer skin of the patient. A protective sheath may be provided around the proximal end 301. This arrangement maintains the position of the proximal end 301 of the guide wire 1228 fixed. It is possible to access the proximal end 301 of the guide wire 1228 at a later time, for example if it is required to alter the location of the treatment element 1201, or to remove the treatment element 1201, for example if the treatment element 1201 became infected. Access may be gained by removing the protective sheath, rotating the guide wire 1228 to unscrew the anchor element 1224 from the ventricle wall, and withdrawing the guide wire 1228 and the treatment element 1201 fixed to the guide wire 1228.

An electrode for pacing of the heart may be provided at the proximal end 301 of the guide wire lead 1228.

It will be appreciated that a variety of possible means may be employed for supporting the treatment element in the desired location at the region of co-aptation of the valve leaflets.

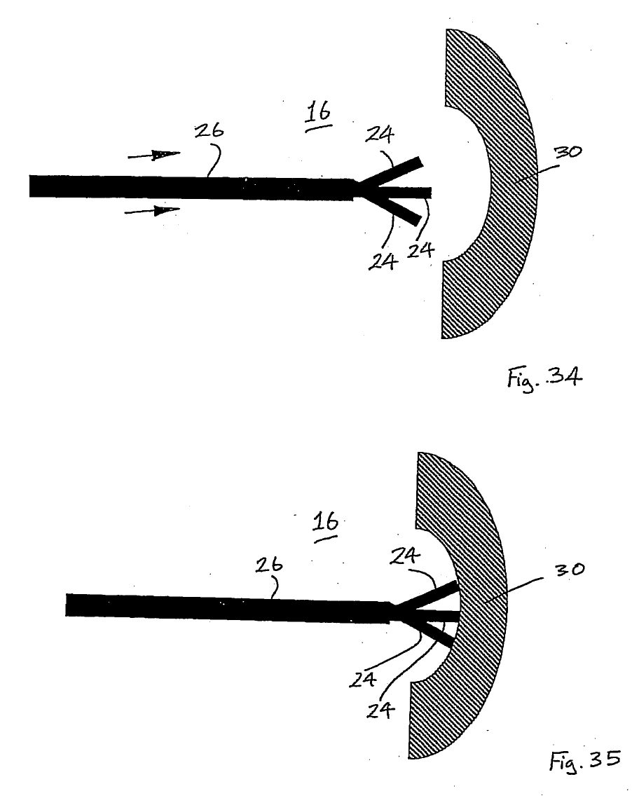

For example the treatment element 20 may be anchored to the septal wall 30 of the ventricle 16 or to the apex of the ventricle 16 by one or more anchor elements 24, with the tether 26 connecting the treatment element 20 to the one or more anchor elements 24. In the medical device illustrated in FIGS. 34 and 35, three anchor elements 24 are employed to anchor the treatment element 20 to the septal wall 30 of the ventricle 16. By increasing the number of anchoring points this arrangement may reduce the degree of trauma at the ventricle wall at each anchor point, and the level of force exerted on the ventricle wall at each anchor point. Thus the possibility of the ventricle wall being damaged, or of the treatment element 20 being dislodged is minimised.

It will be appreciated that the anchor element(s) of the medical device may be anchored to any suitable wall of the heart, and/or to the valve leaflets.

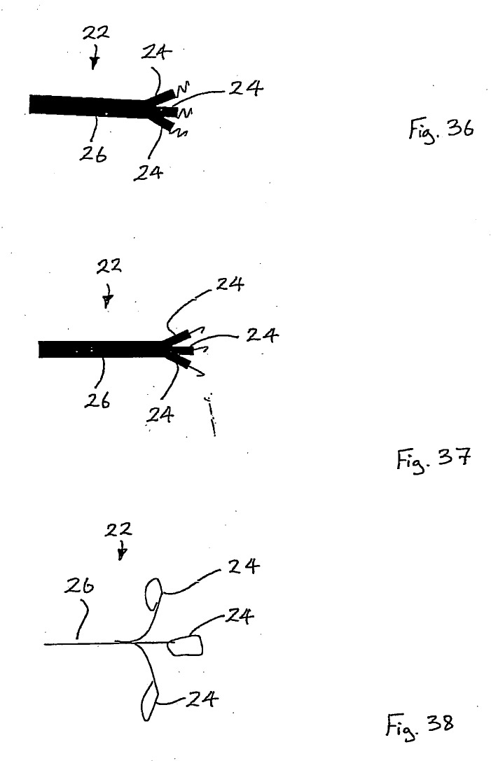

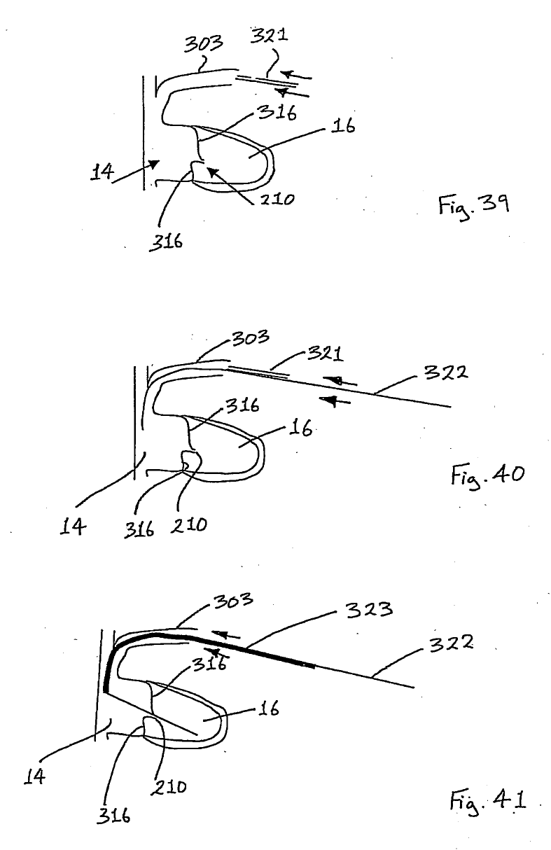

One or more of the anchor elements 24 may be provided in the form of a threaded screw element to anchor to the ventricle wall by rotating the tether 26 to screw the anchor element 24 into the ventricle wall (FIG. 36). Alternatively one or more of the anchor elements 24 may be provided in the form of a hook to anchor to the ventricle wall by hooking into the ventricle wall (FIG. 37). Alternatively one or more of the anchor elements 24 may be provided in the form of a suture loop to anchor to the ventricle wall by suturing to the ventricle wall (FIG. 38).

Referring to FIGS. 39 to 52 there is illustrated a further medical device 310 according to the invention, which is similar to the device of FIGS. 29 and 30, and similar elements in FIGS. 39 to 52 are assigned the same reference numerals.

In this case the medical device 310 comprises the treatment element 311, the delivery system 211 and the support element.

The treatment element 311 is substantially conically shaped with the apex 312 of the cone extending through the valve opening 210 (FIG. 49).

The support element comprises three anchor elements 313 connected to the treatment element 311 by means of three connecting tethers 314, and four support arms 315 protruding radially outwardly from the treatment element 311. Together the anchor elements 313 and the support arms 315 support the treatment element 311 in the desired location at the region of co-aptation of the valve leaflets 316 with the treatment element 311 extending through the valve opening 210. The anchor elements 3 13 anchor the treatment element 3 11 to the septal wall of the ventricle 16 or to the apex of the ventricle 16, and the support arms 315 abut the inner wall of the atrium 14 and the valve leaflets 316 to support the treatment element 311.

In use, the subclavian vein 303 is accessed using a needle 321 (FIG. 39) The procedure may employ a transeptal puncture using a Brockenberg needle. A wire 322 is fed through the needle 321 (FIG. 40). The needle 321 is removed and a sheath 323 is fed over the wire 322 into the ventricle 16 (FIG. 41). The distal end of the sheath 323 is deflectable and can be moved in all planes.

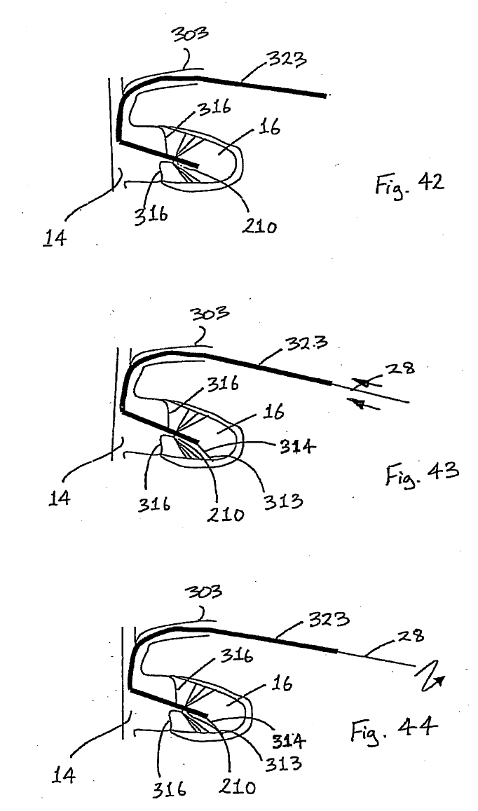

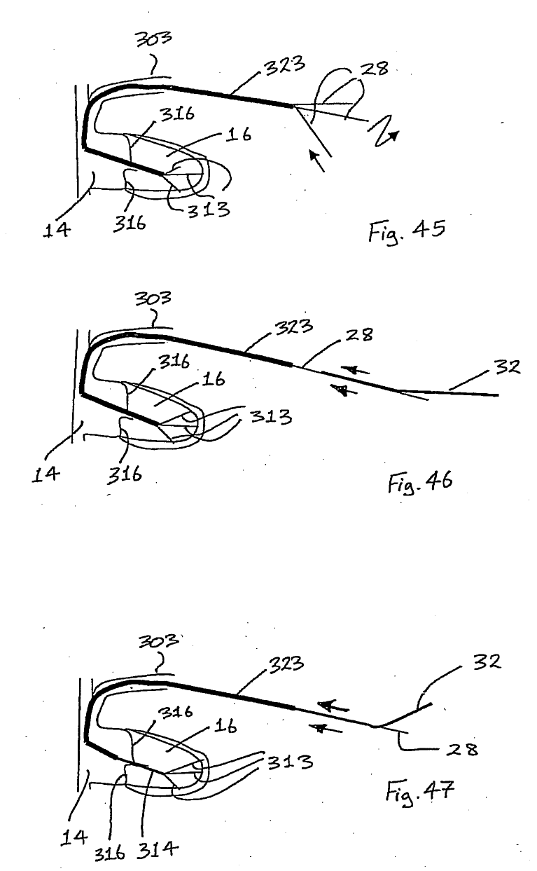

The sheath 323 is used to access the left or right ventricle. For the left ventricle access, a transeptal puncture is performed. The wire 322 is removed (FIG. 42), and the fixation support guide wire 28 is fed through the sheath 323 into the ventricle 16 to abut on the ventricular myocardium (FIG. 43). The wire 28 is rotated in order to screw the support wire 28 into the myocardium by means of the screw anchor elements 313 (FIG. 44). A second and third support wire 28 are fixed in the ventricle 16 in different positions (FIG. 45). By anchoring the treatment element 311 to the ventricle using three anchor elements 313, this assists in evenly distributing the forces exerted on the ventricle. The treatment element 311 in its folded form is delivered through a rapid exchange delivery sheath 32 over the support wire 28 (FIGS. 46 and 47). The treatment element 311 is delivered in the delivery sheath 32 to the correct position using 2D and 3D echo imaging, for example transesophogeal, or transthoracic, or intracardiac, or x-ray, including CT.

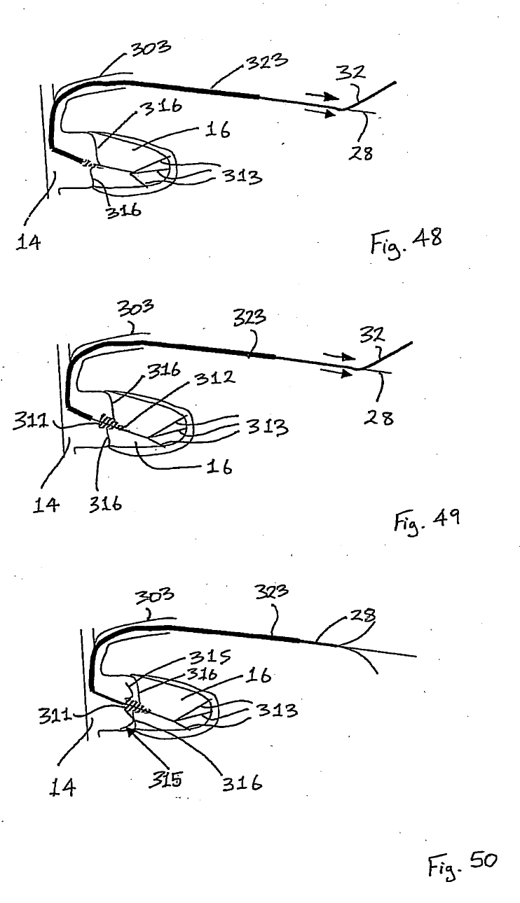

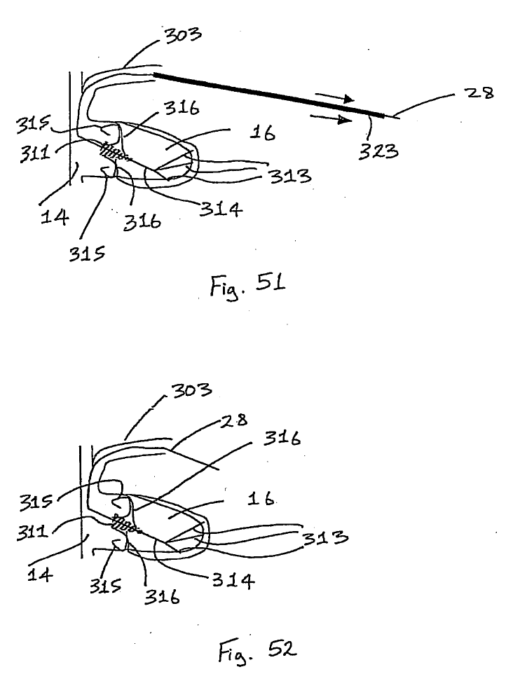

The delivery sheath 32 is withdrawn to deploy the treatment element 311 on the support wire 28 (FIG. 48). The treatment element 311 is fixed on the wire 28 by a spring loaded clamp that is released as the delivery sheath 32 is withdrawn (FIG. 49). The coiled wire supports 315 are delivered into the atrial side of the treatment element 311 to support and maintain the treatment element 311 in the vertical and horizontal plane (FIG. 50). The amount of coiled wire 315 delivered can be varied to alter the position of the treatment element 311.

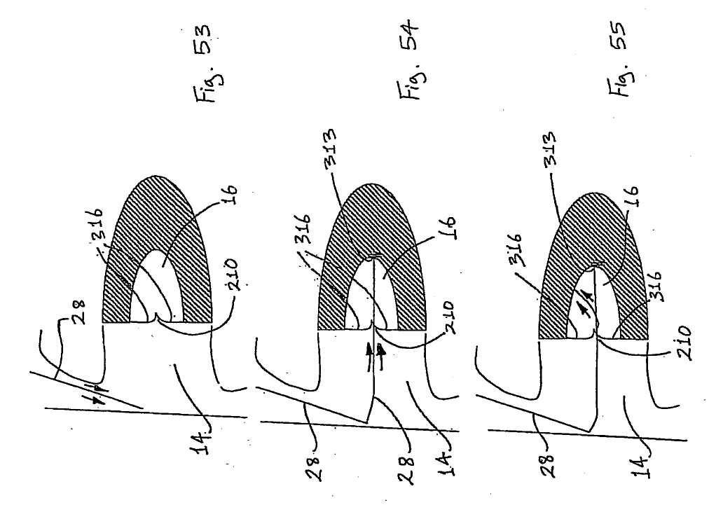

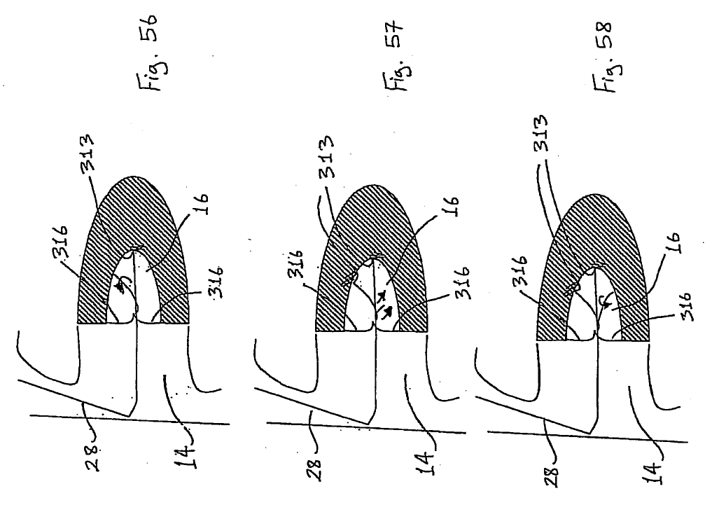

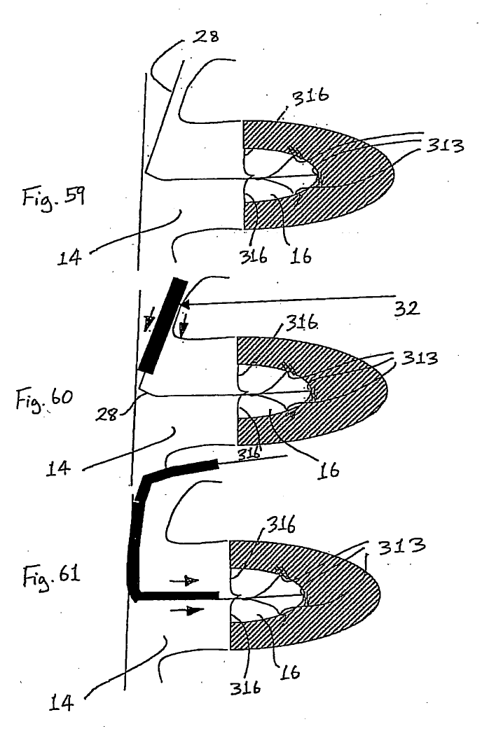

The sheath 323 is withdrawn (FIG. 51), and the redundant wire 28 is cut to length, or has a docking connection to allow extension, and sutured to the subcutaneous tissues before the wound is closed (FIG. 52). This allows re-access to the treatment element 311 for repositioning at a later date, if required.

In FIGS. 53 to 63 there is illustrated another medical device 400 according to the invention, which is similar to the device 310 of FIGS. 39 to 52, and similar elements in FIGS. 53 to 63 are assigned the same reference numerals.

In this case the treatment element 311 is substantially cylindrically shaped. When deployed, the treatment element 311 extends through the valve opening 210 (FIG. 63).

The support element comprises three anchor elements 313 connected to the treatment element 311 by means of three connecting tethers 314 (FIG. 63). The anchor elements 313 support the treatment element 311 in the desired location at the region of co-aptation of the valve leaflets 316 with the treatment element 311 extending through the valve opening 210. The anchor elements 313 anchor the treatment element 311 to the septal wall of the ventricle 16 or to the apex of the ventricle 16.

In use, the fixation support wire guide 28 is fed into the ventricle 16 to abut on the ventricular myocardium (FIG. 53). The wire 28 is rotated in order to screw the support wire 28 into the myocardium by means of the screw anchor elements 313 (FIG. 54). A second and third support wire 28 are fixed in the ventricle 16 in different positions (FIGS. 55 to 59). The treatment element 311 in its folded form is delivered through a rapid exchange delivery sheath 32 over the support wire 28 (FIGS. 60 and 61). The treatment element 311 is delivered in the delivery sheath 32 to the correct position using 2D and 3D echo imaging , for example transesophogeal, or transthoracic, or intracardiac, or x-ray, including CT.

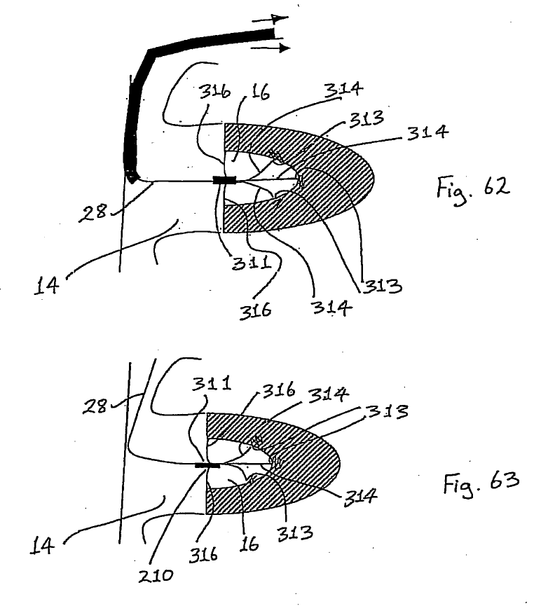

The delivery sheath 32 is withdrawn over the wire 28 to deploy treatment element 311 on the support wire 28 (FIG. 62). The treatment element 311 is fixed in place on the wire 28 by a spring loaded clamp that is released as the delivery sheath 32 is withdrawn (FIG. 62).

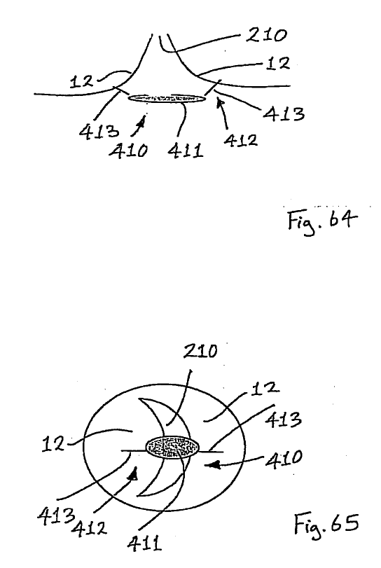

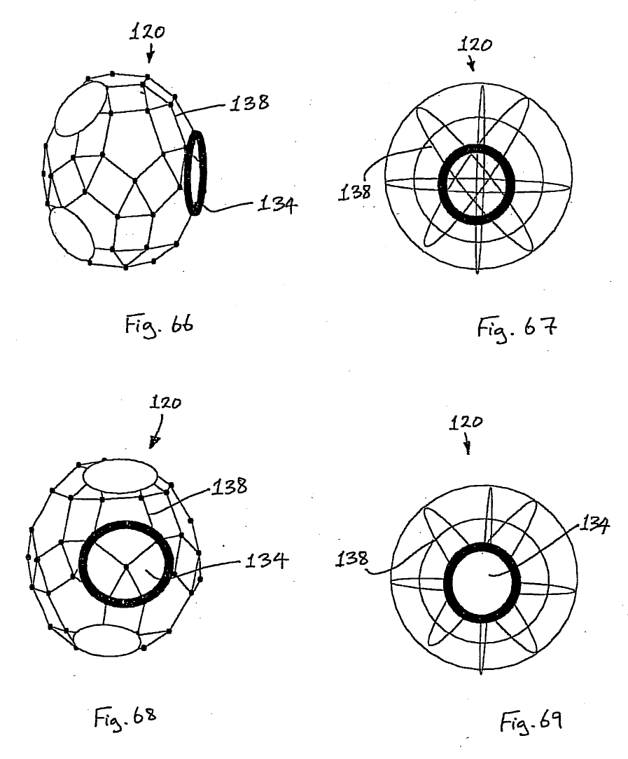

FIGS. 64 and 65 illustrate another medical device 410 according to the invention.

In this case the device 410 comprises the treatment element 411 and the support element 412.

The treatment element 411 comprises a disc element which has a substantially elliptical shape (FIG. 65). The plane of the disc 411 lies substantially perpendicular to the longitudinal axis through the valve opening 210.

The support element 412 comprises two tether arms 413 which are anchored to the valve leaflets 12 to support the treatment element 411 in the desired location at the region of co-aptation of the valve leaflets 12. In this case each tether arm 413 is sutured to a mitral valve leaflet 12.

In this case the treatment element 411 is supported located in the atrium 14 externally of the valve opening 210 (FIG. 64).

Referring now to FIGS. 66 to 74, there is illustrated another medical device 110 according to the invention, which is adapted to occlude a gap at an interface of a pair of valve leaflets 112 of a heart. The device 110 employs the same surgical method as described above with reference to the device 10 of FIGS. 2 to 9. As described with reference to FIGS. 2 to 9, the pair of valve leaflets 112 are located between an atrium 114 and a ventricle 116, and are prevented from deforming outwardly into the atrium 114 by a pair of cordae tendinae 118.

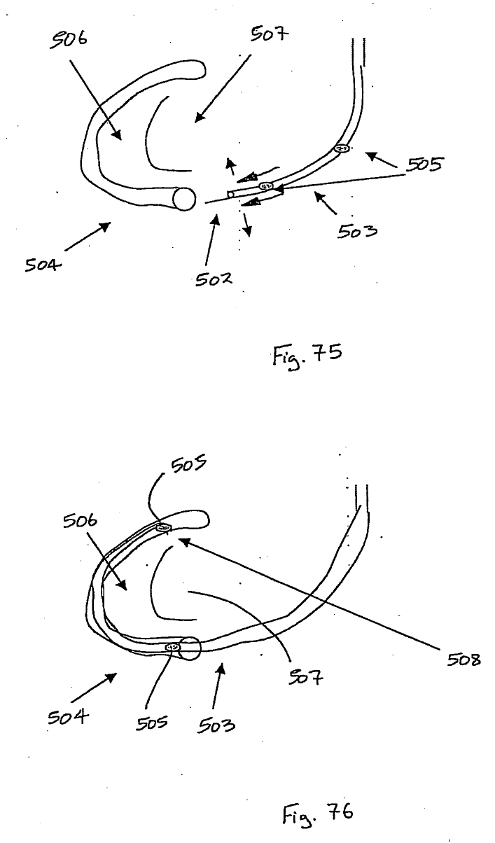

The device 110 comprises a plug 120 which is located, in use, such as to at least partially occlude a gap located at an interface of the pair of valve leaflets 112. However, in this case, the plug 120 is substantially larger in form, and when finally located in position within the atrium 114, is not tethered to the ventricle 116, but acts as its own support in order to secure itself in place. Due to the size of the plug 120, it will be appreciated that the plug 120 should be resiliently deformable in order to be displaceable between a collapsed state (FIG. 71) and an expanded state (FIG. 73), as will be described in detail hereinafter, to facilitate the percutaneous delivery thereof.

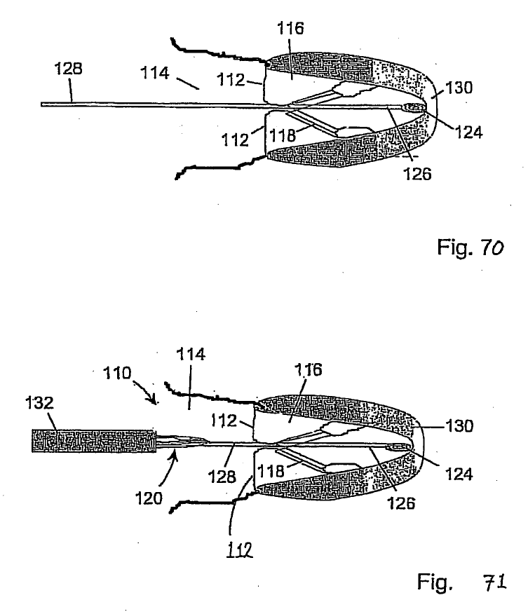

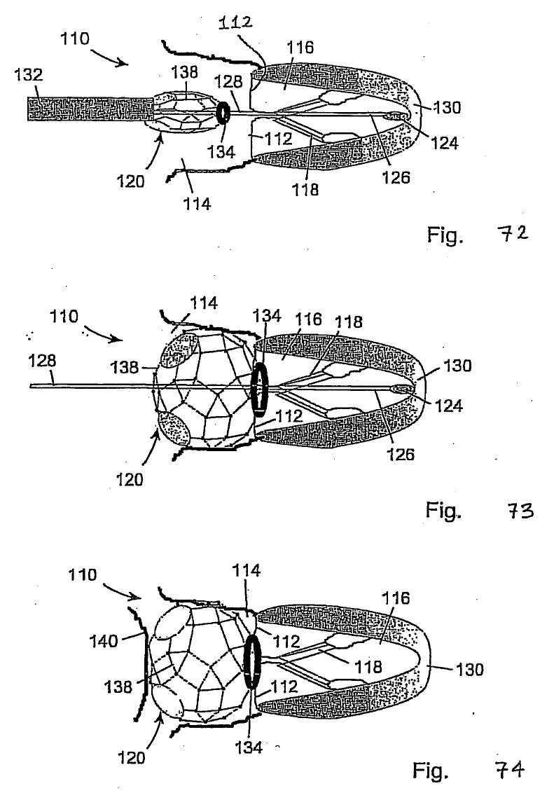

Considering FIG. 70, an anchor 124 is provided at an end of the guide wire 128, which is inserted, percutaneously, into the atrium 114. The guide wire 128 is advanced between the pair of valve leaflets 112, and into contact with a septal wall 130 of the ventricle 116. The guide wire 128 is then rotated about a longitudinal axis thereof, in order to thread the anchor 124 into the septal wall 130.

Turning to FIG. 71, a sheath or catheter 132 is advanced over the guide wire 128 until the free end of the catheter 132 is in communication with the atrium 114. The plug 120 is then advanced through the catheter 132, in the collapsed state, into the atrium 114, as illustrated in FIG. 72. Once the plug 120 has been advanced fully out of the catheter 132, the plug 120 will automatically displace into the expanded state wherein the catheter 132 can be removed, as illustrated in FIG. 73. The plug 120 is hollow in form, but carries a substantially fluid impermeable contact surface 134 thereon, which in use is positioned against the valve leaflets 112, in order to at least partially support the valve leaflets 112 and/or at least partially occlude the gap therebetween.

The plug 120, being hollow, is comprised of a substantially spherical shell 138 which is reticulated in form, and which provides dual functionality to the plug 120. The reticulated nature of the shell 138 enables the plug 120 to be deformable between the collapsed and expanded state, in addition to allowing the free flow of blood into and through the plug 120, other than through the contact surface 134, in order to allow blood to flow between the atrium 114 and the ventricle 116 when the plug 120 is present. The resiliently deformable nature of the plug 120 also allows the slight deformation thereof as the atrium 114 itself deforms during pumping of blood into the ventricle 116.

The plug 120 is dimensioned such that, when deployed in the atrium 114, the plug 120 contacts both the valve leaflets 112 and a back wall 140 of the atrium 114 (FIG. 74), in order to ensure that the plug 120 is sufficiently supported in position within the atrium 114. As a result once the plug 120 is located in position, the anchor 124 may be unscrewed from the septal wall 130, and the guide wire 128 withdrawn from the heart, as illustrated in FIG. 74. The plug 120 is left in place within the atrium 114, with the fluid impermeable contact surface 134 seated against the interface between the pair of valve leaflets 112. It will be appreciated that the plug 120, in supporting the contact surface 134 in position, takes the place of the support 22 of the device 10 of FIGS. 2 to 9.

It will be appreciated that the plug could be of any other suitable form once the functionality thereof is retained, namely to be capable of being seated between or against the valve leaflets in order to at least partially occlude a gap therebetween, thereby substantially or completely preventing the regurgitation of blood. For example, a plug having a conical or cylindrical contact surface could be employed, which could then be inserted partially or wholly within the gap between the valve leaflets.

It will be appreciated that any suitable means may be employed in order to deliver the plug into position, and any suitable means may also be employed to secure the plug in position once delivered.

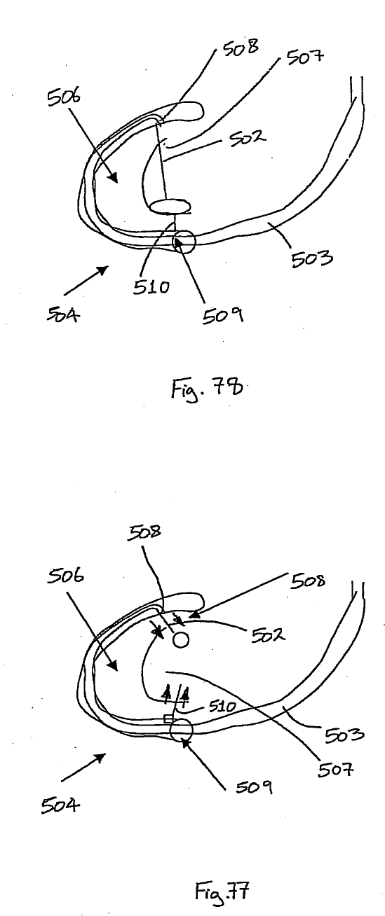

Referring to FIGS. 75 to 80, there is illustrated a further medical device 500 according to the invention, which is similar to the device 410 of FIGS. 64 and 65. In this case the treatment element 501 is deployed in the desired location by initially inserting a wire 502 and a deflectable catheter 503 over the wire 502 into the coronary sinus 504. Side holes 505 are provided in the catheter 503 to facilitate coronary sinus puncture (FIG. 75).

The posterior mitral valve leaflet 506 and the anterior mitral valve leaflet 507 are also illustrated in FIG. 75.

The catheter 503 is inserted into the coronary sinus 504, and a small puncture 508 is made from the anterior coronary sinus 504 to the left atrium and the catheter 503 is inserted into the left atrium (FIG. 76). The coronary sinus 504 is used to guide the catheter 503.

The looped wire 502 is fed into the left atrium through the first puncture 508, and a sheath is advanced over the wire 502 (FIG. 77). A second puncture 509 is made from the inferior coronary sinus 504 to the left atrium and a grasp 310 is fed into the second puncture 509 to grab the looped wire 502 (FIG. 77).

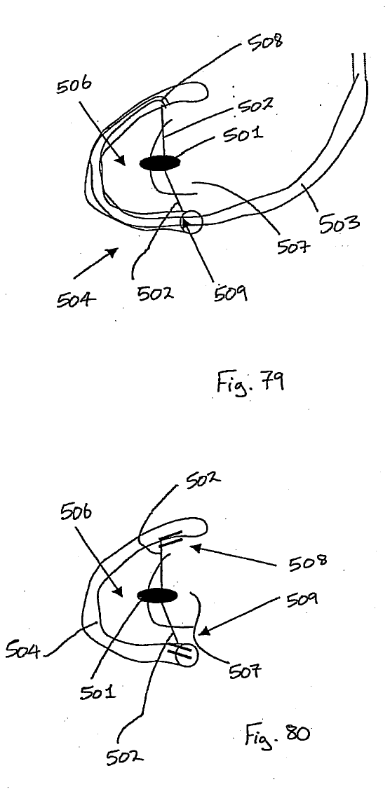

The looped wire 502 is pulled into the second puncture 509 to deliver the treatment element 501 and fix the treatment element 501 in the desired location across the mitral valve at the region of co-aptation of the valve leaflets 506, 507 (FIG. 78).

FIG. 79 illustrates the treatment element 501 pulled into position across the mitral valve. The treatment element 501 is supported in the desired location at the region of co-aptation of the valve leaflets by means of clamping the support wires 502, 510 into position at the coronary sinus 504/left atrium punctures 508, 509 (FIG. 80).

The invention is not limited to the embodiments described herein, with reference to the accompanying drawings, which may be amended or modified in construction and detail without departing from the scope of the present invention.

1-125. (canceled)

126. A device for the treatment of a valve defect, the device comprising:
a treatment element; and
a treatment wire;
the treatment element having an expanded treatment configuration and a collapsed delivery configuration;
the treatment wire having a distal end, a proximal end, a distal segment, a transition segment, and a proximal segment;
the treatment device comprising an anchor at a distal end;
the treatment element being slidable relative to the treatment wire and being lockable to the treatment wire.

127. The device as claimed in claim 126 wherein the proximal segment of the treatment wire is detachable from the distal segment of the treatment wire.

128. The device as claimed in claim 126 wherein the proximal segment of the treatment wire is configured to be located exterior to a patient.

129. The device as claimed in claim 127 wherein the transition segment is adjacent a point of detachment of the proximal segment.

130. The device as claimed in claim 126 wherein the transition segment is adapted to provide an atraumatic tissue implant interface.

131. The device as claimed in claim 130 wherein the atraumatic tissue implant interface comprises a soft polymeric interface, or a porous interface, or a mechanical stress-distributing element.

132. The device as claimed in claim 126 wherein the device comprises a locking element for locking the treatment element to the treatment wire.

133. A device for the treatment of a valve defect, the device comprising:
a treatment element; and
a treatment wire;
the treatment element having an expanded treatment configuration and a collapsed delivery configuration;
the treatment wire having a distal end, a proximal end and a proximal segment;
an anchor at the distal end;
the treatment element being connected to the treatment wire proximal of the distal end of the treatment wire.

134. The device as claimed in claim 133 wherein the treatment element is advanceable through a procedure catheter.

135. The device as claimed in claim 134 wherein the treatment element is advanceable through a procedure catheter having a deflectable tip.

136. The device as claimed in claim 133 wherein the treatment wire is a wire, or a tube, or a combination of a wire and a tube.

137. The device as claimed in claim 133 wherein the treatment wire is at least partially metallic or polymeric.

138. The device as claimed in claim 133 wherein the treatment wire comprises an outer jacket and an inner core.

139. The device as claimed in claim 138 wherein the outer jacket is polymeric and the inner core is metallic.

140. The device as claimed in claim 138 wherein the core is translatable or rotatable relative to the outer jacket.

141. The device as claimed in claim 138 wherein the inner core is engagable with the anchor at the distal end of the treatment wire.

142. The device as claimed in claim 138 wherein relative movement of the core is configured to anchor the anchor in a wall of a heart.

143. The device as claimed in claim 142 wherein the core is movable relative to the outer jacket to anchor the anchor in a wall of a heart.

144. The device as claimed in claim 138 wherein the inner core is removable from the outer jacket.

145. The device as claimed in claim 138 wherein the outer jacket is a non-thrombogenic polymer.

146. The device as claimed in claim 138 wherein the outer jacket is coated or covered with a non-thrombogenic coating, and/or a drug eluting coating, and/or a coating containing an active agent, and/or an active agent and/or a drug.

147. The device as claimed in claim 133 wherein the treatment element is a self-expanding element.

148. The device as claimed in claim 133 wherein the treatment element is expandable by inflation.

149. The device as claimed in claim 133 wherein the treatment wire comprises a multi lumen tubing.

150. The device as claimed in claim 149 wherein at least one lumen is an inflation lumen.

151. The device as claimed in claim 150 wherein the inflation lumen is occludable after inflation.

152. The device as claimed in claim 151 wherein the inflation lumen is occludable using a soft polymeric interface as a proximal plug or valve.

153. The device as claimed in claim 133 wherein the treatment element is expandable by mechanical actuation.

154. The device as claimed in claim 138 wherein the core is a pacing lead.

155. The device as claimed in claim 133 wherein the treatment element is slidable over the treatment wire in the collapsed configuration and is coupled to the wire in the expanded configuration.

156-221. (canceled)

222. A method of treating a valve using a treatment device, the treatment device comprising a treatment element, a treatment wire and an anchor element, the method comprising the steps of:
advancing a procedural catheter into the atrium;
advancing the treatment wire through the procedural catheter and passing the distal end of the treatment wire across the valve;
anchoring a distal end of the treatment wire to a wall of the ventricle;
expanding the treatment element at the desired region; and
terminating the proximal end of the wire beneath the skin of the patient.

223. The method as claimed in claim 222 wherein the method comprises the step of steering the procedural catheter to allow ease of advancement of the treatment device.

224. The method as claimed in claim 223 wherein the steering step comprises torqueing a shaped procedural catheter.

225. (canceled)

226. The method as claimed in claim 222 wherein the method comprises the step of collapsing the treatment element.

227. The method as claimed in claim 222 wherein the anchoring step comprises a relative motion between a core of the treatment wire and an outer tube of the treatment wire.

228. The method as claimed in claim 227 wherein the anchoring step relative motion comprises torqueing the core relative to the outer tube to anchor a distal end of the treatment wire.

229. The method as claimed in claim 227 wherein the anchoring step relative motion comprises advancing the core relative to the outer tube to anchor a distal end of the treatment device.

230. The method as claimed in claim 222 wherein the method comprises the step of inserting the collapsed treatment element into the procedural catheter.

231. The method as claimed in claim 222 wherein the method comprises the step of advancing the treatment element over at least a portion of the treatment wire.

232. The method as claimed in claim 222 wherein the step of terminating the proximal end of the treatment wire comprises the step of removing a proximal segment of the wire or removing the inner core.

233. The method as claimed in claim 232 wherein the step of removing a proximal end of the treatment wire comprises cutting, and/or unscrewing, and/or decoupling, and/or cutting, and/or breaking the proximal end of the wire.

234. The method as claimed in claim 232 wherein the step of terminating the proximal end of the treatment wire comprises engaging a soft cap with the end of the wire.

235. The method as claimed in claim 232 wherein the step of terminating the proximal end of the treatment wire comprises closing the puncture site with the proximal end of the treatment wire beneath the skin.

236. The method as claimed in claim 222 wherein the method comprises the step of locking the treatment element to the treatment wire adjacent the valve.

237. The method as claimed in claim 226 the step of collapsing the treatment element comprises the step of loading the treatment element into a delivery catheter distal end.

238. The method as claimed in claim 237 wherein the method comprises the step of advancing the treatment element and the delivery catheter over the treatment wire.

239. The method as claimed in claim 237 where the method of advancing the catheter comprises a rapid exchange technique.

240. The method as claimed in claim 237 wherein the step of expanding the treatment element comprises retraction of the sheath relative to a fixing abutment.

241. The method as claimed in claim 222 wherein the method comprises the step of adjusting the position of the treatment device relative to the valve.

242. The method as claimed in claim 241 wherein the position adjusting step comprises visualising the treatment device under fluoroscopy using radiopaque markers on the treatment device.

243. The method as claimed in claim 241 wherein the position adjusting step comprising visualising the treatment device using an ultrasound probe and ultrasound visible markers positioned on the treatment device.

244-268. (canceled)
